(19) United States
(12) Patent Application Publication
(10) Pub. No.: US 2023/0032409 A1
(43) Pub. Date: Feb. 2, 2023

(54) Method for Information Encoding and Decoding, and Method for Information Storage and Interpretation

(71) Applicants: BGI SHENZHEN, Shenzhen, Guangdong (CN); BGI SHENZHEN CO., LIMITED, Shenzhen, Guangdong (CN)

(72) Inventors: Shihong CHEN, Shenzhen, Guangdong (CN); Xiaoluo HUANG, Shenzhen, Guangdong (CN); Zhi PING, Shenzhen, Guangdong (CN); Tao LIN, Shenzhen, Guangdong (CN); Chen CHAI, Shenzhen, Guangdong (CN); Yue SHEN, Shenzhen, Guangdong (CN); Xun XU, Shenzhen, Guangdong (CN); Huanming YANG, Shenzhen, Guangdong (CN)

(21) Appl. No.: 17/763,221

(22) PCT Filed: Sep. 24, 2019
(86) PCT No.: PCT/CN2019/107426

§ 371 Date: Mar. 24, 2022

(51) Int. Cl. G06F 3/06

(52) U.S. Cl. CPC G06F 3/0655; G06F 3/0604; G06F 3/0673

(57) ABSTRACT

A method for information encoding and decoding, and method for information storage and interpretation are provided. The information encoding method includes: first binary information and second binary information as well as a first encoding rule and a second encoding rule are obtained; a first output candidate symbol corresponding to a current input of the first binary information is obtained and a second output candidate symbol corresponding to a current input of the second binary information is obtained, and an intersection of the first output candidate symbol and the second output candidate symbol is taken as an output corresponding to a current input; and an output symbol corresponding to each binary bit of the first binary information and the second binary information is sequentially determined through the first encoding rule and the second encoding rule, as to obtain an encoding sequence formed by a plurality of the output symbols.

This is a continuation of International Application No. PCT/CN2019/107426 filed on Sep. 24, 2019, the disclosure of which is incorporated herein by reference in its entirety.

### TECHNICAL FIELD

The present disclosure relates to the technical field of information storage, and in particular to an information encoding method and apparatus, an information decoding method and apparatus, a storage medium, and an information storage and interpretation method.

### BACKGROUND ART

With the development of modern technology, especially the Internet, global data shows an exponential increase. The ever-increasing amount of the data puts forward higher and higher requirements on storage technologies. Traditional storage technologies, such as tape and optical disk storage, are increasingly unable to meet the current data storage requirements due to limited storage density and time. In recent years, the development of a DNA storage technology provides a new way to solve these problems. Compared with traditional storage media, DNA as a medium for information storage has the characteristics of long storage time (it can be stored for more than thousands of years, which is more than a hundred times that of the tape and optical disk medium known by the inventor) and has a high storage density (the storage density is up to about 109 Gb/mm3, which is ten million times more than that of the tape and optical disk medium) and good storage security and the like.

DNA data storage usually includes the following steps: 1) encoding: a binary 0/1 code of computer information is converted into DNA sequence information of an A/T/C/G base; 2) synthesizing: a DNA synthesis technology is used to synthesize a corresponding DNA sequence, and an obtained DNA molecule is stored in an in vitro medium or a living cell; 3) sequencing: a sequencing technology is used to read the DNA sequence of the stored DNA molecule; and 4) decoding: a method corresponding to the encoding process in Step 1) is used to convert the DNA sequence obtained by sequencing into the binary 0/1 code, and further convert into the computer information.

In order to achieve the effective DNA data storage, it is necessary to develop a technology for the above processes. Herein, the encoding and decoding technologies involved in Steps 1) and 4) are the most critical technology for the DNA data storage. The most critical parts of this technology as follows: 1) How to maximize the density of 0/1 binary information encoded by DNA. The improvement in DNA storage density is essential to save the cost of DNA synthesis for storing information in Step 2). 2) While the 0/1 binary information is converted into the A/T/C/G base sequence, the single-base repetition, high GC and high AT between the sequences are avoided to the greatest extent. Generally speaking, continuous single-base repetition appears in the DNA sequence, both the high GC and high AT can cause difficulty in reading sequence information during the sequencing process. A conversion method of the 0/1 binary information and the A/T/C/GDNA sequence directly determines the difficulty of reading the DNA sequence during the sequencing process, and thus determines the fidelity of the data in the reading process.

At present, the most classic DNA data storage methods include a data encoding method proposed by George Church and Goldman et al. George Church et al. proposed the most primitive conversion method of binary 0/1 information to A/T/C/G base information in 2012, namely, 0 stands for A/T, 1 stands for C/G, and the encoding of 1 binary data by each nucleotide is achieved. This method can avoid the continuous single-base repetition to a certain extent, but it cannot avoid high GC or high AT in the data. While a large number of 0 or 1 repetitions appear in the data, the encoded DNA sequence may produce continuous GC or AT. At the same time, in this encoding mode, each nucleotide encodes only 1 bit of information, which is very limited.

Goldman et al. proposed a new DNA encoding method in 2013, which improved the storage density of DNA data to a certain extent. It firstly converts binary data into a ternary 0/1/2 data through Huffman coding, and then converts the 0/1/2 data into a “quaternary” A/T/C/G sequence through a designed rule. This encoding method also avoids the continuous single-base repetition, but it is still limited in the storage density of the DNA data, and its maximum coding density is 1.6 bits/base (nt). At the same time, this encoding method cannot completely solve the situation of continuous GC or AT appearing in the coding sequence. Although some other encoding methods are different from the above two methods, they cannot completely avoid the continuous GC and AT situations or the storage density is still limited.

In order to use DNA more effectively for the binary data storage, improve the density of the DNA data storage, and avoid the continuous GC and AT situations, it is particularly important to develop more effective DNA data encoding methods.

### SUMMARY

The present invention provides an information encoding method and apparatus, an information decoding method and apparatus, a storage medium, and an information storage and interpretation method. Two pieces of input binary information are converted and integrated as one piece of information, the information capacity limit is reached, and the information encoding density is high.

According to a first aspect, a method for information encoding is provided in an embodiment, including the following operations:

First binary information and second binary information as well as a first encoding rule and a second encoding rule are obtained, wherein the first encoding rule is used for encoding the first binary information, and the second encoding rule is used for encoding the second binary information.

A first output candidate symbol corresponding to a current input of the first binary information is obtained according to the first encoding rule, and a second output candidate symbol corresponding to a current input of the second binary information is obtained according to the second encoding rule, and an intersection of the first output candidate symbol and the second output candidate symbol is taken as an output symbol corresponding to a current input.

An output symbol corresponding to each binary bit of the first binary information and the second binary information is sequentially determined through the first encoding rule and the second encoding rule, as to obtain an encoding sequence formed by a plurality of the output symbols.

In a preferred embodiment, the first output candidate symbols are two of four symbols, the second output candidate symbols are two of the four symbols, and the first output candidate symbols and the second output candidate symbols have one same symbol.

The above first encoding rule is that, under a support bit of the first encoding rule, two of the four symbols are selected as the first output candidate symbols while the current input of the first binary information is 0, and the other two are used as the first output candidate symbol while the current input is 1, herein the support bit of the above first encoding rule is any one of the four symbols, and the support bit of the above first encoding rule has a first corresponding relationship with a current output bit.

The above second encoding rule is that, under the support bit of the first encoding rule and a support bit of the second encoding rule, two of the four symbols are selected as the second output candidate symbols while the current input of the second binary information is 0, the other two are used as the second output candidate symbols while the current input is 1, herein the support bit of the above second encoding rule is any one of the four symbols, and the support bit of the above second encoding rule has a second corresponding relationship with the current output bit, each support bit of the first encoding rule corresponds to four support bits of the second encoding rule.

In a preferred embodiment, a length of the above first binary information and a length of the above second binary information are equal.

In a preferred embodiment, the above first binary information and second binary information are split from a same piece of binary information.

In a preferred embodiment, the above first corresponding relationship is a first predetermined number of bits before the above current output bit; and the second corresponding relationship is a second predetermined number of bits before the current output bit.

In a preferred embodiment, the above method further includes: an initiation base sequence is obtained, and it provides the support bit for the first encoding rule and the second encoding rule before an above output base is generated.

In a preferred embodiment, the above first output candidate symbol are two base symbols of four base symbols composed of A, T, C, and G, and the above second output candidate symbol are two base symbols of the four base symbols composed of A, T, C, and G, the above support bit is one base symbol of the four base symbols composed of A, T, C, and G.

In addition, the above encoding information is a nucleic acid sequence containing the above four bases composed of A, T, C, and G.

In a preferred embodiment, the above method further includes: an initiation sequence is obtained, and it provides an initiation support bit for the first encoding rule and the second encoding rule before a first bit of the above encoding sequence is generated.

In a preferred embodiment, the above method further includes: before the first binary information and the second binary information are obtained, the above first binary information and second binary information are extracted from a computer storage device.

According to a second aspect, an apparatus for information encoding is provided in an embodiment, including an information acquiring unit, an information encoding unit, and a result generating unit.

The information acquiring unit is configured to obtain first binary information and second binary information as well as a first encoding rule and a second encoding rule, herein the first encoding rule is used for encoding the first binary information, and the second encoding rule is used for encoding the second binary information.

The information encoding unit is configured to obtain a first output candidate symbol corresponding to a current input of the first binary information according to the first encoding rule, and obtain a second output candidate symbol corresponding to a current input of the second binary information according to the second encoding rule, and take an intersection of the first output candidate symbol and the second output candidate symbol as an output symbol corresponding to the current input.

The result generating unit is configured to sequentially determine an output symbol corresponding to each binary bit of the first binary information and the second binary information through the first encoding rule and the second encoding rule, as to obtain an encoding sequence formed by a plurality of the above output symbols.

In a preferred embodiment, the above first output candidate symbols are two of four symbols, the above second output candidate symbols are two of the four symbols, and the above first output candidate symbols and the second output candidate symbols have one same symbol.

The above first encoding rule is that, under a support bit of the first encoding rule, two of the four symbols are selected as the first output candidate symbols while the current input of the first binary information is 0, and the other two are used as the first output candidate symbols while the current input is 1, herein the support bit of the above first encoding rule is any one of the four symbols, and the support bit of the above first encoding rule has a first corresponding relationship with a current output bit.

The above second encoding rule is that, under the support bit of the first encoding rule and a support bit of the second encoding rule, two of the four symbols are selected as the second output candidate s symbols while the current input of the second binary information is 0, the other two are used as the second output candidate symbols while the current input is 1, wherein the support bit of the above second encoding rule is any one of the four symbols, and the support bit of the above second encoding rule has a second corresponding relationship with the current output bit, each support bit of the first encoding rule corresponds to four support bits of the second encoding rule.

In a preferred embodiment, the above first corresponding relationship is a first predetermined number of bits before the above current output bit; and the second corresponding relationship is a second predetermined number of bits before the above current output bit.

In a preferred embodiment, the above first output candidate symbols are two base symbols of four bases composed of A, T, C, and G, and the above second output candidate symbol are two base symbols of the four base symbols composed of A, T, C, and G, the above support bit is one base symbol of the four base symbols composed of A, T, C, and G. In addition, the above encoding information is a nucleic acid sequence containing the above four bases composed of A, T, C, and G.

According to a third aspect, a computer-readable storage medium is provided in an embodiment, including a program, and the program may be executed by a processor to achieve the method as described in the first aspect.

According to a fourth aspect, a method for information storage by using a DNA sequence is provided in an embodiment, including the following operations:

Through the information encoding method as described in the first aspect, binary information to be stored is converted into DNA sequence information, and the above DNA sequence information includes a base sequence formed by four bases composed of A, T, C, and G.

A corresponding DNA sequence is synthesized according to the above DNA sequence information.

The above DNA sequence is saved to achieve the storage of information.

In a preferred embodiment, the above method further includes: The above DNA sequence information is split into multiple pieces of DNA short sequence information, and a DNA index sequence identifier is added to each piece of the split DNA short sequence information, and the above DNA index sequence identifier includes position order information of the above DNA short sequence information.

A corresponding DNA sequence is synthesized according to the above DNA short sequence information.

The above DNA sequence is saved to achieve the storage of information.

In a preferred embodiment, the above DNA sequence is saved in the form of dry powder, or saved by embedding in an embedding material.

In a preferred embodiment, the above DNA sequence is transferred into a living cell for saving.

In a preferred embodiment, the above living cell is a microbial cell, preferably Escherichia coli or Saccharomyces cerevisiae.

According to a fifth aspect, a method for information decoding is provided in an embodiment, including the following operations:

An encoding sequence generated by the above encoding method as described in the first aspect, as well as a first encoding rule and a second encoding rule are obtained, herein the first encoding rule is used for encoding first binary information, and the second encoding rule is used for encoding second binary information.

A current symbol of the above encoding sequence is read, and according to a corresponding relationship between four different symbols and the binary information in the first encoding rule and the second encoding rule, the above current symbol is converted into a binary bit of the first binary information and the second binary information.

Through a corresponding relationship between the different symbols and the binary information in the first encoding rule and the second encoding rule, each binary bit of the first binary information and the second binary information corresponding to each symbol bit of the above encoding sequence is determined sequentially, and the first binary information and the second binary information with a determined binary bit order are obtained.

In a preferred embodiment, the above symbol is four base symbols composed of A, T, C, and G.

In a preferred embodiment, the above encoding sequence is obtained through the following steps (1) or (2):

(1) Each DNA sequence synthesized by the storage method as described in the fourth aspect is sequenced, as to obtain the above encoding sequence; or

(2) each DNA sequence synthesized by the storage method as described in the fourth aspect is sequenced, as to obtain each DNA short sequence information; according to a DNA index sequence identifier, position order information of each DNA short sequence is obtained; and according to the above position order information, the above each DNA short sequence is combined to form the above encoding sequence.

In a preferred embodiment, the above decoding method further includes the following operation:

The above first binary information and second binary information are transcoded into corresponding information.

In a preferred embodiment, the above corresponding information is text information, image information, audio information and/or video information.

According to a sixth aspect, an apparatus for information decoding is provided in an embodiment, including an information acquiring unit, an information decoding unit and a result generating unit.

The information acquiring unit is configured to obtain an encoding sequence generated by the encoding apparatus as described in the second aspect, as well as a first encoding rule and a second encoding rule, herein the first encoding rule is used for encoding first binary information and the second encoding rule is used for encoding second binary information.

The information decoding unit is configured to read a current symbol of the above encoding sequence, and according to a corresponding relationship between different symbols and binary information in the first encoding rule and the second encoding rule, convert the above current symbol into a binary bit of the first binary information and the second binary information.

The result generating unit is configured to, through a corresponding relationship between four different symbols and the binary information in the first encoding rule and the second encoding rule, sequentially determine each binary bit of the first binary information and the second binary information corresponding to each symbol bit of the above encoding sequence, and obtain the first binary information and the second binary information with a determined binary bit order.

In a preferred embodiment, the above different symbols are four base symbols composed of A, T, C, and G respectively.

In a preferred embodiment, the above encoding sequence is obtained by the following units (1) or (2):

(1) A sequencing unit is configured to sequence each DNA sequence synthesized by the above storage method as described in the fourth aspect, as to obtain the above encoding sequence; or

(2) A sequencing unit is configured to sequence each DNA sequence synthesized by the above storage method as described in the fourth aspect, as to obtain each DNA short sequence information.

An index unit is configured to obtain position order information of above each DNA short sequence according to a DNA index sequence identifier.

A combination unit is configured to combine the above each DNA short sequence into the above encoding sequence according to the position order information.

In a preferred embodiment, the above apparatus further includes a transcoding unit. The transcoding unit is configured to transcode the above first binary information and second binary information into corresponding information.

In a preferred embodiment, the above corresponding information is text information, image information, audio information and/or video information.

According to a seventh aspect, a computer-readable storage medium is provided in an embodiment, including a program, and the program may be executed by a processor to achieve the decoding method as described in the fifth aspect.

The methods for information encoding and decoding provided by the present invention may convert and integrate two pieces of input binary information into one piece of information, the information capacity limit is reached, and the information encoding density is high. In a preferred embodiment, the four base symbols composed of A, T, C, and G are used as four different symbols to convert and integrate the two pieces of the input binary information into one DNA sequence skillfully, so that the limit of information capacity of a DNA single-base reaches 2 bits/base, and the information encoding density is high.

In practical applications, the method of the present invention may be well combined with long-segment gene storage to improve the DNA storage density. The method of the present invention utilizes two encoding rules to generate 5566277615616 encoding systems, provides a rich compilation method rule library for DNA storage applications, and greatly expands a choice space of DNA storage compilation methods.

At the same time, the encoding rule in the method of the present invention depends on the two support bits, so that the encoding mode has the higher tolerance for repeated input, and may effectively avoid or reduce the impact caused by continuous single-base and double-base repetitions. In addition, the encoding rule library generated by the method of the present invention utilizes different encoding rule combinations for encrypted storage, increases the difficulty of decrypting the DNA storage information, improves the security of the DNA storage information well, and provides the guarantee for future data security storage applications.

### BRIEF DESCRIPTION OF THE DRAWINGS

FIG. 1 is a general process diagram of DNA storage and interpretation information in an embodiment of the present invention.

FIG. 2 is a process diagram of an encoding method for converting binary information into a DNA sequence in an embodiment of the present invention.

FIG. 3 is a schematic diagram of a general representation method of an encoding rule in an embodiment of the present invention.

FIG. 4 is a schematic diagram of a method for representing a case of the encoding rule in an embodiment of the present invention.

FIG. 5 is a schematic diagram of a principle of converting one piece of binary information into a DNA sequence in an embodiment of the present invention.

FIG. 6 is a structure block diagram of an encoding apparatus for converting binary information into a DNA sequence in an embodiment of the present invention.

FIG. 7 is a process diagram of a decoding method for converting a DNA sequence into binary information in an embodiment of the present invention.

FIG. 8 is a structure block diagram of a decoding apparatus for converting a DNA sequence into binary information in an embodiment of the present invention.

FIG. 9 is a process diagram of a method for information storage by using a DNA sequence in an embodiment of the present invention.

FIG. 10 is a process diagram of a method for interpreting information stored in the form of a DNA sequence in an embodiment of the present invention.

### DETAILED DESCRIPTION OF THE EMBODIMENTS

The present invention is further described in detail below through specific embodiments in combination with drawings. In the following embodiments, many detailed descriptions are used to enable the present invention to be better understood. However, those skilled in the art may easily realize that some of features may be omitted under different circumstances, or may be replaced by other materials or methods.

In addition, the features, operations, or characteristics described in the description may be combined in any appropriate manners to form various embodiments. At the same time, various steps or actions in the method description may also be sequentially exchanged or adjusted in a manner apparent to those skilled in the art. Therefore, various orders in the description and the drawings are only for the purpose of clearly describing a certain embodiment, and are not meant to be a necessary order, unless it is specified otherwise that a certain order must be followed.

In this article, serial numbers assigned to the characteristics, such as “first”, and “second”, are only used to distinguish described objects, and do not have any order or technical meanings.

In this article, a first encoding rule is also referred to as “encoding rule 1”, and the two have equivalent meanings; a second encoding rule is also referred to as “encoding rule 2”, and the two have equivalent meanings.

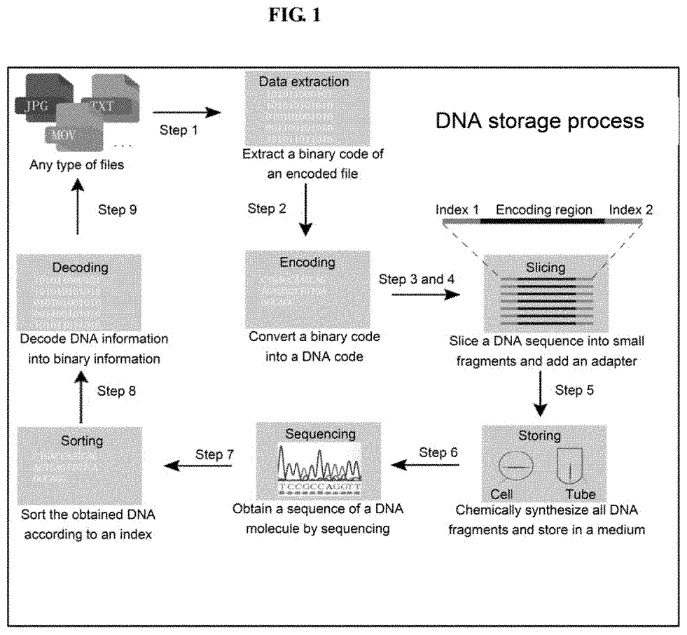

As shown in FIG. 1, the data encoding, storage and interpretation processes of the present invention includes the following steps:

(1) Data Encoding and Storage Process:

Step 1: binary “0/1” computer information to be stored is extracted by using a program that comes with any computer operating systems or a program specially written to extract a binary 0/1 code.

Step 2: an encoding rule in a set of the methods for encoding and decoding in the present invention is used to convert two pieces of binary “0/1” information into a DNA sequence represented by A/T/C/G bases. Generally speaking, the two pieces of the binary “0/1” information have the same length, and the length is greater than or equal to 1.

Step 3: the DNA sequence obtained from the 0/1 binary computer information conversion is split into short fragments with a certain length, and it is convenient for the DNA synthesis in the next step.

Step 4: an index sequence (index 1 and index 2 in the figure) is added to each DNA short fragment, and the index sequence may encode order information of the short fragment obtained in Step 3.

Step 5: a DNA synthesis technology is used to synthesize the DNA sequence fragments obtained in Step 4, and save in a corresponding medium. Generally speaking, the DNA sequence may be saved in a sample tube in the form of dry powder, or saved by embedding in an embedding material such as amber, and a silicon ball, or the DNA sequence may be transferred into a living cell for saving, and the living cell may be a microbial cell, more preferably Escherichia coli or Saccharomyces cerevisiae.

(2) DNA Data Interpretation Process:

Step 6: a sequencing technology (for example, Sanger sequencing or high-throughput sequencing) is used to sequence the saved DNA fragments of storage data information, as to obtain DNA sequences of these fragments.

Step 7: according to index sequence information preset in the DNA sequences, an order of DNA fragment encoding information is interpreted, and the DNA fragments are sorted sequentially.

Step 8: according to a sequence of a DNA fragment encoding information area obtained by sorting, a decoding rule corresponding to the encoding rule in Step 2 is used to convert DNA information into 0/1 binary information.

Step 9: the 0/1 binary information obtained in Step 8 is converted into stored information (namely files, such as a text, an image, an audio or a video) by using a program that comes with any operating systems or a program specially written to convert 0/1 into data information.

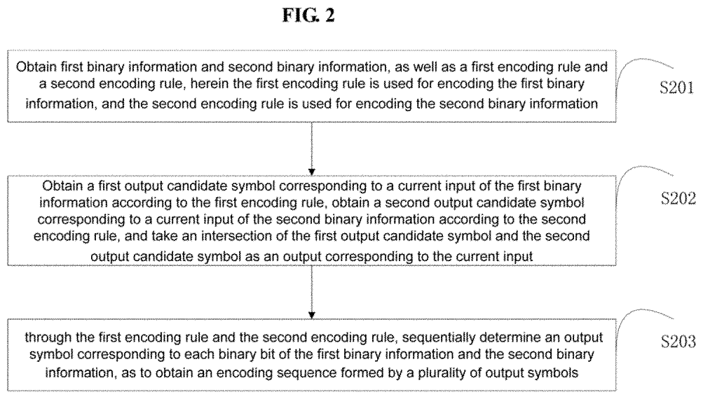

As shown in FIG. 2, an encoding method for converting binary information into a DNA sequence in an embodiment of the present invention includes the following steps:

S201: first binary information and second binary information as well as a first encoding rule and a second encoding rule are obtained, herein the first encoding rule is used for encoding the first binary information, and the second encoding rule is used for encoding the second binary information.

In the embodiment of the present invention, the first binary information and the second binary information are 0/1 binary information to be encoded. Both the first binary information and the second binary information may have the same or different sources, namely, the two may be related 0/1 binary information or unrelated 0/1 binary information. In an example of the related 0/1 binary information, two pieces of the 0/1 binary information are binary information which is split from the same piece of the binary information. It is generally required that the two pieces of the 0/1 binary information have the same length, because in the method of the present invention, one binary bit (0/1) of each piece of the 0/1 binary information read every time is from a pair of binary bits (0/1) of the two pieces of the 0/1 binary information, and converted into a symbol by the method of the present invention, for example, base symbol (A, T, G, or C) information. In an example of the unrelated 0/1 binary information, for example, it is two pieces of the 0/1 binary information from text information and pattern information respectively.

In the embodiment of the present invention, the first encoding rule is an encoding rule of a corresponding relationship between a binary symbol 0/1 and an output symbol (for example, base symbols A, T, G, or C). As a typical but non-deterministic example, a case of the first encoding rule is that, under a support bit of the first encoding rule, two of the four bases composed of A, T, C, and G are selected as a first output candidate base while a current input of the first binary information is 0, and the other two are used as a first output candidate base while the current input is 1.

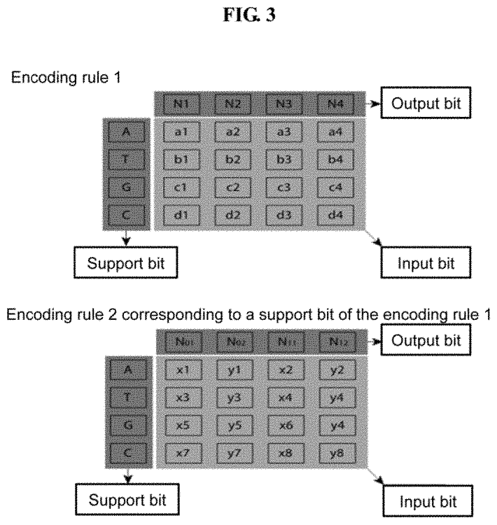

As shown in FIG. 3, vertical rows A, T, G, and C represent the support bits of the four different bases, and N1, N2, N3, and N4 represent one of the four bases composed of A, T, C, and G respectively; a1, a2, a3, and a4 are 0 or 1, and a1+a2+a3+a4-2; b1, b2, b3, and b4 are 0 or 1, and b1+b2+b3+b4=2; c1, c2, c3 and c4 are 0 or 1, and c1+c2+c3+c4=2; and d1, d2, d3, and d4 are 0 or 1, and d1+d2+d3+d4=2. That is to say, in a series a, there are two 1 and two 0. Similarly, there are two 1 and two 0 in series b, c, and d respectively. Therefore, the encoding rule 1 (first encoding rule) has a total of [2 of 4 combinations are chosen, namely “2 of 4 bases are optionally chosen, and each support bit corresponds to 6 possible combinations” ] to the 4-th power types, namely, 6\hat{ }4 or 1296 types, because the base selection between the 4 different support bits is independent of each other. This encoding rule is interpreted as: while the support bit is Nx (x=1, 2, 3, 4) and an input bit is 0/1, N of the two output bits corresponding to 0/1 is the possibility of output.

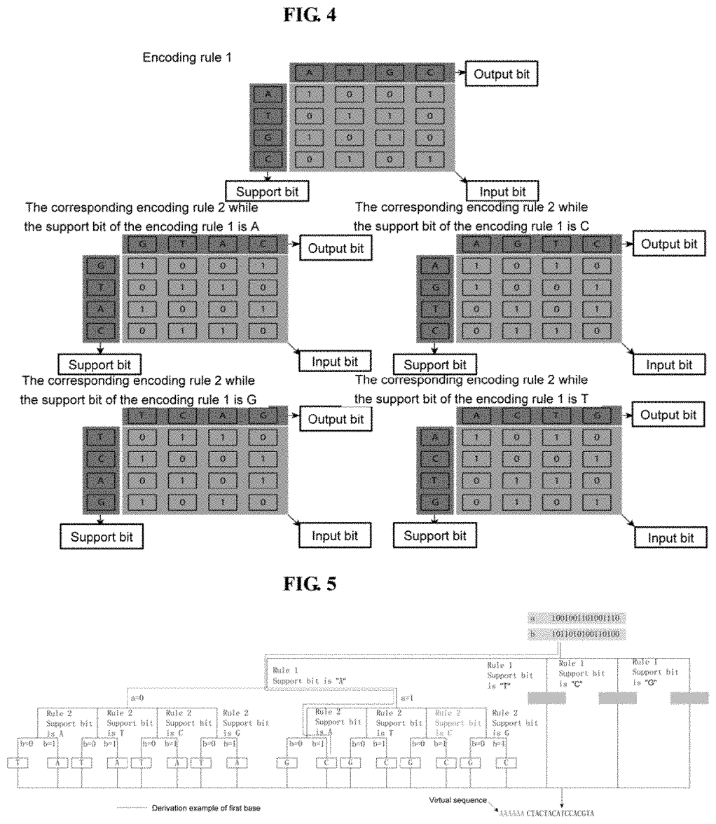

As shown in FIG. 4, a case of the encoding rule 1 is shown. In this case, in the case of the different support bits of the encoding rule 1, the input bit of the first binary information corresponds to a corresponding output bit in a manner shown in the figure.

In the embodiment of the present invention, the input bit refers to a binary bit of the current input of the first binary information (or the second binary information), and it is 0/1; and the output bit refers to a base bit to be output after being converted according to the first encoding rule (or the second encoding rule) corresponding to the input bit.

In the embodiment of the present invention, the support bit of the first encoding rule refers to support information needed for selecting a correct output symbol by the first encoding rule according to different inputs (the so-called “input” refers to the binary bit of the current input of the first binary information) of the first binary information. For example, while the output symbol is the base symbol A, T, C or C, the support bit of the first encoding rule is also the base symbol. Specifically, the support bit of the first encoding rule is a known base that has a first corresponding relationship with the current output bit. Generally speaking, the support bit is base information that has been converted before, for example, the number of bits before a data bit (current output bit) is being converted, such as the previous base information of the 3-th or 6-th bit. Therefore, in an embodiment of the present invention, the so-called “first corresponding relationship” refers to a first predetermined number of bits before the current output bit (for example, the number of bits set arbitrarily such as the 3-th or 6-th bit before the current input bit). Certainly, the support bit may also be virtual information generated randomly, and it has the artificially set first corresponding relationship with the current output bit. In the embodiment of the present invention, the so-called “random generation” means that a random number is generated through various random methods, and corresponds to ATCG according to a certain rule. Examples of the random methods include but are not limited to: Monte Carlo random number or U(0,1) random number and the like. In addition, the support bit may also come from a specific selection mode of a reference sequence, and it has a specific first corresponding relationship with the current output bit. For example, each output bit corresponds to a known base on the reference sequence in a specific mapping manner, such as a base order of the reference sequence corresponds to each output bit sequentially. In the embodiment of the present invention, while the support bits are different, the same input may have different base outputs.

In the embodiment of the present invention, the second encoding rule is an encoding rule of a corresponding relationship between a binary symbol 0/1 and an output symbol (for example, a base symbol A, T, G, or C). As a typical but non-deterministic example, a case of the second encoding rule is that, under the support bit of the first encoding rule and the support bit of the second encoding rule, two of the four bases are selected as second output candidate bases while an input of the second binary information is 0, and the other two are used as second output candidate bases while the input is 1; and wherein the support bit of the second encoding rule is any one of the four bases, and the support bit of the second encoding rule has a second corresponding relationship with the current output bit, and each support bit of the first encoding rule corresponds to four support bits of the second encoding rule.

As shown in FIG. 3, N01 and N02 are two possible bases while the input in the encoding rule 1 is 0, and N11 and N12 are two possible bases while the input in the encoding rule 1 is 1. Because in the encoding rule 1, the different support bits thereof may have different output possibilities, while the support bits in the encoding rule 1 are different, the corresponding encoding rule 2 (second encoding rule) may be different. Wherein xn+yn=1, xn*yn=0, n=1, 2, 3, 4, 5, 6, 7, 8. In other words, there is one 0 and one 1 in each x and y group. Therefore, corresponding to the possibility given in each encoding rule 1, there are 28 types of the encoding rule 2. This is because the base selection between the different support bits of the encoding rule 2 is independent, and the encoding rule 2 corresponding to each support bit of each encoding rule 1 has (2\hat{ }2)\hat{ }4 possibilities, namely 256 possibilities. This encoding rule is interpreted as that while the support bit is Nx (x=01, 02, 11, 12) and the input bit is 0/1, N of the two output bits corresponding to 0/1 is the possibility of output.

As shown in FIG. 4, a case of the encoding rule 2 is shown. In this case, in the case of the different support bits of the encoding rule 1, it is respectively shown that under the different support bits of the encoding rule 2, the input bit of the second binary information corresponds to a corresponding output bit in the manner shown in the figure. Specifically, it shows the corresponding encoding rule 2 while the support bit of the encoding rule 1 is A, the corresponding encoding rule 2 while the support bit of the encoding rule 1 is C, the corresponding encoding rule 2 while the support bit of the encoding rule 1 is C, and the corresponding encoding rule 2 while the support bit of the encoding rule 1 is T.

In the embodiment of the present invention, similar to the support bit of the first encoding rule, the support bit of the second encoding rule refers to support information needed for selecting a correct output symbol by the second encoding rule according to different inputs (the so-called “input” refers to the binary bit of the current input of the second binary information) of the second binary information. For example, while the output symbol is the base symbol A, T, G, or C, the support bit of the second encoding rule is also the base symbol. Specifically, the support bit of the second encoding rule is a known base that has a second corresponding relationship with the current output bit. Generally speaking, the support bit is a base that has been converted before, for example, the number of bits before a data bit (current output bit) is being converted, such as the previous base information of the 4-th or 8-th bit. Therefore, in an embodiment of the present invention, the so-called “second corresponding relationship” refers to a second predetermined number of bits before the current output bit (for example, the previous number of bits set arbitrarily such as the 4-th or 8-th bit). Certainly, the support bit may also be virtual information generated randomly, and it has the artificially set second corresponding relationship with the current output bit. In the embodiment of the present invention, the so-called “random generation” means that a random number is generated through various random methods, and corresponds to ATCG according to a certain rule. Examples of the random methods include but are not limited to: Monte Carlo random number or U(0,1) random number and the like. In addition, the support bit may also come from a specific selection mode of a reference sequence, and it has a specific second corresponding relationship with the current output bit. For example, each output bit corresponds to a known base on the reference sequence in a specific mapping manner, such as a base order of the reference sequence corresponds to each output bit sequentially. In a preferred embodiment of the present invention, the first corresponding relationship is different from the second corresponding relationship, namely the support bit of the first encoding rule and the support bit of the second encoding rule take different base bits. For example, in an embodiment of the present invention, the 6-th known base before the current output bit is selected as the support bit of the first encoding rule, and the first known base before the current output bit is selected as the support bit of the second encoding rule.

Since the base selection of the different support bits in the encoding rule 1 is independent, and the base selection of the different support bits in the encoding rule 2 is also independent, the number of the types of the encoding rule 2 corresponding to each encoding rule 1 is 256\hat{ }4, namely, 4294967296 types, so the total number of the types of this dual-encoding rule system is 1296*4294967296, namely 5566277615616, and it is about 5.6×10\hat{ }12 types.

In this way, one binary input bit (0/1) from the first binary information obtains two possible lists of output through the encoding rule 1, and one binary input bit (0/1) from the second binary information obtains two possible lists of output through the encoding rule 2, an intersection of the two output lists is taken, thereby an output base corresponding to the two input binary bits is determined.

It should be noted that in the case of using the base information that has been converted before as the support bit, while the conversion is initially started, since the base information that has been converted before as the support bit does not exist, an initial support bit problem needs to be solved by an appropriate method. In an embodiment of the present invention, the encoding method of the present invention further includes: a initiation base sequence is obtained, and it provides the support bit for the first encoding rule and the second encoding rule before the output base is generated. In other cases, for example, in the case of using the virtual information generated randomly, or the specific selection mode from the reference sequence as the support bit, such a problem does not exist.

S202: a first output candidate symbol corresponding to a current input of the first binary information is obtained according to the first encoding rule, and a second output candidate symbol corresponding to a current input of the second binary information is obtained according to the second encoding rule, and an intersection of the first output candidate symbol and the second output candidate symbol is taken as an output symbol corresponding to a current input.

In the embodiment of the present invention, as a preferred example, the first output candidate symbols and the second output candidate symbols are two base symbols in four bases composed of A, T, C, and G respectively. Therefore, the first output candidate symbol and the second output candidate symbol refer to a first output candidate base and a second output candidate base, respectively.

In the embodiment of the present invention, the so-called “current input” refers to a 0/1 binary bit currently being read from the first binary information or the second binary information, and it represents one binary data respectively. The present invention reads the 0/1 binary bits in the first binary information and the second binary information at the same time. While one 0/1 binary bit of the first binary information is read, one 0/1 binary bit of the second binary information is read at the same time. One 0/1 binary bit read of the first binary information is converted into the first output candidate base (including two bases) through the first encoding rule, and one 0/1 binary bit read of the second binary information is converted into the second output candidate base (including two bases) through the second encoding rule, and there is a common base between the first output candidate base and the second output candidate base, namely an intersection of the first output candidate base and the second output candidate base, and the intersection is the output base corresponding to the two current inputs (the current input of the first binary information and the current input of the second binary information).

S203: an output symbol corresponding to each binary bit of the first binary information and the second binary information is sequentially determined through the first encoding rule and the second encoding rule, as to obtain an encoding sequence formed by a plurality of the above output symbols.

Since the first binary information and the second binary information each have a certain length, for example, tens, hundreds, thousands, or tens of thousands of bits (0/1 binary bit), each time of the conversion operation in the above step S202 may only convert information of one bit (0/1 binary bit) in the first binary information and the second binary information into the output base respectively, so the conversion operation needs to be continuously performed, until the information of all bits (0/1 binary bit) in the first binary information and the second binary information are converted into the corresponding output bases, and a plurality of such output bases forms the corresponding DNA sequence. So far, the conversion of two pieces of the 0/1 binary information (the first binary information and the second binary information) into a piece of the DNA sequence information is completed.

In order to enable the present invention to be understood more easily, as a typical but non-deterministic example, as shown in FIG. 5: there are two pieces of binary information a and b, and a 0/1 binary information encoding sequence thereof is shown in FIG. 5. A virtual sequence shown in the figure is used as a initiation base support bit, the ninth binary bit 1 of the binary information a is converted into a G/C base while the support bit is A according to the first encoding rule (Table 1); and while the support bit of the ninth binary bit 1 of the binary information a is A, and the support bit of the ninth binary bit 1 of the binary information b is A, the ninth binary bit 1 of the binary information b is converted into a A/C base while the support bit is A according to the second encoding rule (Table 2), and an intersection of the G/C base and the A/C base is taken, namely the base C is used as the output base.

Those skilled in the art may understand that all or part of the functions of the various methods in the above embodiments may be achieved in the form of hardware, or achieved in the form of a computer program. While all or part of the functions in the above embodiments is achieved in the form of the computer program, the program may be stored in a computer-readable storage medium. The storage medium may include: a read-only memory, a random access memory, a magnetic disk, an optical disk, a hard disk and the like. The program is executed by a computer to achieve the above functions. For example, the program is stored in a memory of a device, and while the program in the memory is executed by a processor, all or part of the above functions may be achieved. In addition, when all or part of the functions in the above embodiments is achieved in the form of the computer program, the program may also be stored in the storage medium such as a server, another computer, a magnetic disk, an optical disk, a flash disk, or a mobile hard disk, and saved in a memory of a local device by downloading or copying, or the version of a system of the local device is updated, while the program in the memory is executed by the processor, all or part of the functions in the above embodiments may be achieved.

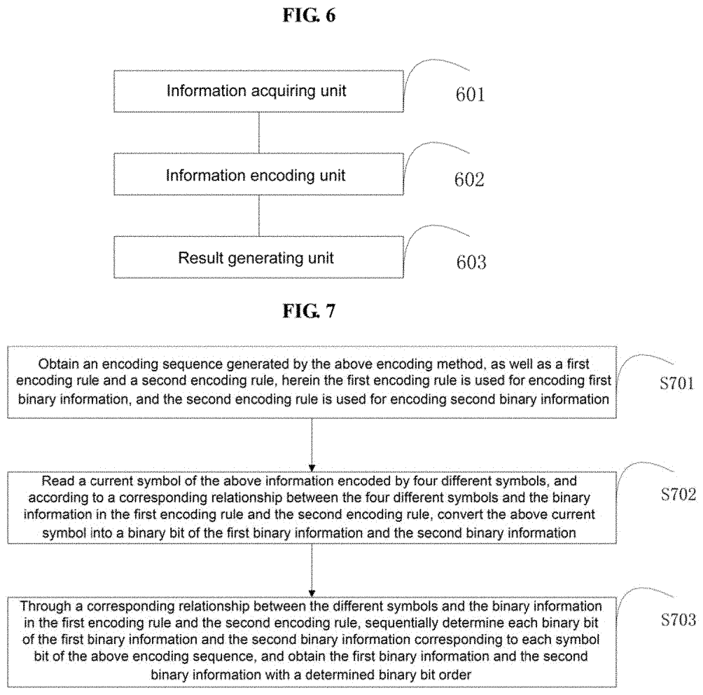

Therefore, corresponding to the information encoding method of the present invention, an embodiment of the present invention further provides an apparatus for information encoding. As shown in FIG. 6, the apparatus includes: an information acquiring unit 601, configured to obtain first binary information and second binary information as well as a first encoding rule and a second encoding rule, herein the first encoding rule is used for encoding the first binary information, and the second encoding rule is used for encoding the second binary information; an information encoding unit 602, configured to obtain a first output candidate symbol corresponding to a current input of the first binary information according to the first encoding rule, and obtain a second output candidate symbol corresponding to a current input of the second binary information according to the second encoding rule, and take an intersection of the first output candidate symbol and the second output candidate symbol as an output symbol corresponding to the current input; and a result generating unit 603, configured to sequentially determine an output symbol corresponding to each binary bit of the first binary information and the second binary information through the first encoding rule and the second encoding rule, as to obtain an encoding sequence formed by a plurality of the above output symbols.

In a preferred embodiment of the present invention, the above first output candidate symbols and second output candidate symbols are two base symbols of four bases composed of A, T, C, and G respectively; the above first encoding rule is that, under a support bit of the first encoding rule, two of the four bases composed of A, T, C, and G are selected as the first output candidate base while the current input of the first binary information is 0, and the other two are used as the first output candidate base while the current input is 1; the second encoding rule is that, under the support bit of the first encoding rule and a support bit of the second encoding rule, two of the four bases are selected as the second output candidate base while the current input of the second binary information is 0, the other two are used as the second output candidate base while the current input is 1; and wherein the support bit of the first encoding rule is a known base that has a first corresponding relationship with the current output bit, the support bit of the second encoding rule is a known base that has a second corresponding relationship with the current output bit, and each support bit of the first encoding rule corresponds to four support bits of the second encoding rule.

Corresponding to the information encoding method of the present invention, an embodiment of the present invention further provides a computer-readable storage medium, including a program, and the program may be executed by a processor to achieve the information encoding method as described in the present invention.

As an inverse process of the encoding method of the present invention, an embodiment of the present invention further provides an information decoding method. As shown in FIG. 7, the method includes:

S701: an encoding sequence generated by the above encoding method, as well as a first encoding rule and a second encoding rule are obtained, herein the first encoding rule is used for encoding first binary information, and the second encoding rule is used for encoding second binary information.

In the embodiment of the present invention, the encoding sequence generated by the encoding method, for example, may be a piece of DNA sequence information. Correspondingly, as a typical but non-deterministic example, the first encoding rule is that: under the support bit of the first encoding rule, two of the four bases composed of A, T, C, and G are selected as the first output candidate bases while the input of the first binary information is 0, and the other two are used as the first output candidate bases while the input is 1.

As a typical but non-deterministic example, in the embodiment of the present invention, the second encoding rule is that: under the support bit of the first encoding rule and the support bit of the second encoding rule, two of the four bases are selected as the second output candidate bases while the input of the second binary information is 0, and the other two are used as the second output candidate bases while the input is 1.

In the embodiment of the present invention, the support bit of the first encoding rule is a known base that has a first corresponding relationship with the current output bit, and the support bit of the second encoding rule is a known base that has a second corresponding relationship with the current output bit, and each support bit of the first encoding rule corresponds to four support bits of the second encoding rule.

S702: a current symbol of the above information encoded by four different symbols is read, and according to a corresponding relationship between the four different symbols and the binary information in the first encoding rule and the second encoding rule, the above current symbol is converted into a binary bit of the first binary information and the second binary information.

As a typical but non-deterministic example, in the embodiment of the present invention, the four bases composed of A, T, C, and G are used as four different encoding symbols. Correspondingly, the current symbol is also called “current base”. The so-called “current base” refers to a base on a DNA sequence that is currently read and converted into the binary bit. Since there are tens, hundreds, thousands, or even tens of thousands of the bases in the DNA sequence, each base being currently read and converted is the so-called “current base”.

S703: through a corresponding relationship between the different symbols and the binary information in the first encoding rule and the second encoding rule, each binary bit of the first binary information and the second binary information corresponding to each symbol bit of the above encoding sequence is determined sequentially, and the first binary information and the second binary information with a determined binary bit order are obtained.

In an embodiment of the present invention, the above encoding sequence is obtained through the following steps (1) or (2).

(1) Each DNA sequence synthesized by the storage method of the present invention is sequenced, as to obtain the above encoding sequence; or

(2) each DNA sequence synthesized by the storage method of the present invention is sequenced, as to obtain each DNA short sequence information; according to a DNA index sequence identifier, position order information of each DNA short sequence is obtained; and according to the above position order information, the above each DNA short sequence is combined to form the above encoding sequence.

In an embodiment of the present invention, the above decoding method further includes: the above first binary information and second binary information are transcoded into corresponding information, for example, text information, image information, audio information and/or video information.

It should be noted that many details in the above decoding method, especially the details related to the technical features such as the first binary information, the second binary information, the first encoding rule, the second encoding rule, the input bit, the output bit, and the support bit, are the same as the details of such technical features in the above encoding method, so they are not repeatedly described.

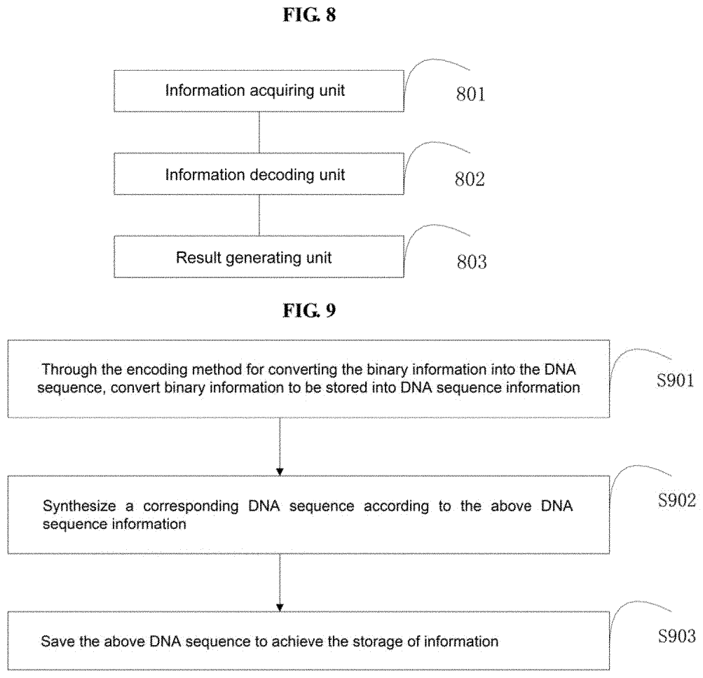

Corresponding to the information decoding method of the present invention, an embodiment of the present invention further provides an apparatus for information decoding. As shown in FIG. 8, the apparatus includes: an information acquiring unit 801, configured to obtain an encoding sequence generated by the above encoding apparatus, as well as a first encoding rule and a second encoding rule, herein the first encoding rule is used for encoding first binary information, and the second encoding rule is used for encoding second binary information; an information decoding unit 802, configured to read a current symbol of the above encoding sequence, and according to a corresponding relationship between different symbols and binary information in the first encoding rule and the second encoding rule, convert the above current symbol into a binary bit of the first binary information and the second binary information; and a result generating unit 803, configured to, through a corresponding relationship between four different symbols and the binary information in the first encoding rule and the second encoding rule, sequentially determine each binary bit of the first binary information and the second binary information corresponding to each symbol bit of the above encoding sequence, and obtain the first binary information and the second binary information with a determined binary bit order.

In an embodiment of the present invention, the above encoding sequence is obtained by the following units (1) or (2).

(1) A sequencing unit is configured to sequence each DNA sequence synthesized by the storage method of the present invention, as to obtain the above encoding sequence; or

(2) a sequencing unit is configured to sequence each DNA sequence synthesized by the storage method of the present invention, as to obtain each DNA short sequence information; an index unit is configured to obtain position order information of above each DNA short sequence according to a DNA index sequence identifier; and a combination unit is configured to combine the above each DNA short sequence into the above encoding sequence according to the position order information.

In an embodiment of the present invention, the above decoding apparatus further includes a transcoding unit. The transcoding unit is configured to transcode the above first binary information and second binary information into corresponding information, for example, text, image, audio or video information.

In an embodiment of the present invention, the above different symbols are the four base symbols composed of A, T, C, and G; and similarly, many details in the above decoding device, especially the details related to the technical features such as the first binary information, the second binary information, the first encoding rule, the second encoding rule, the input bit, the output bit and the support bit, are the same as the details of such technical features in the above encoding method, so they are not repeatedly described.

Corresponding to the information decoding method of the present invention, an embodiment of the present invention further provides a computer-readable storage medium, including a program, and the program may be executed by a processor to achieve the information decoding method as described in the present invention.

An embodiment of the present invention further provides a method for information storage by using a DNA sequence. As shown in FIG. 9, the method includes the following operations.

S901: through the information encoding method of the present invention, binary information to be stored is converted into DNA sequence information, and the DNA sequence information includes a base sequence formed by four bases composed of A, T, C, and G.

S902: a corresponding DNA sequence is synthesized according to the above DNA sequence information.

S903: the above DNA sequence is saved to achieve the storage of information.

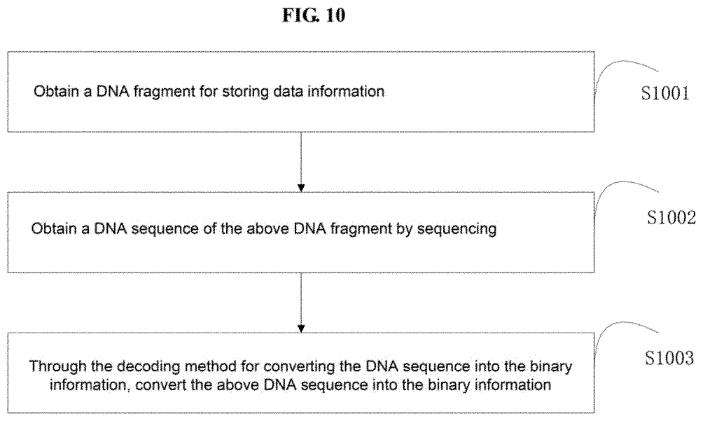

An embodiment of the present invention further provides a method for interpreting information stored in the form of a DNA sequence. As shown in FIG. 10, the method includes the following operations.

S1001: a DNA fragment for storing data information is obtained.

S1002: a DNA sequence of the above DNA fragment is obtained by sequencing.

S1003: through the decoding method for converting the DNA sequence into the binary information, the above DNA sequence is converted into the binary information.

In some embodiments, the DNA sequence information converted from the binary information is longer, and it is not beneficial to direct synthesis. Therefore, as a preferred method, the DNA sequence information is split into a plurality of pieces of DNA short sequence information, and a DNA index sequence identifier is added to each piece of the split DNA short sequence information, and the above DNA index sequence identifier contains position order information of the above DNA short sequence information; then, the corresponding DNA sequence is synthesized according to the above DNA short sequence information; and finally, the above DNA sequence is saved to achieve the storage of information.

Generally speaking, the DNA sequence may be saved in a sample tube in the form of dry powder, or saved by embedding in an embedding material such as amber, and a silicon ball, or the DNA sequence may be transferred into a living cell for saving, and the living cell may be a microbial cell, more preferably Escherichia coli or Saccharomyces cerevisiae.

In some embodiments, there are a plurality of pieces of the DNA fragments for storing the data information, and each piece of the DNA fragment has a corresponding index sequence as the encoding order information of the DNA fragment. In this case, in order to obtain the complete binary information, the DNA sequence obtained by sequencing needs to be sorted firstly according to the index sequence to obtain a complete DNA sequence after sorting, and then the complete DNA sequence is converted into the binary information through the decoding method of the present invention for converting the DNA sequence into the binary information.

The technical schemes of the present invention are described in detail below through the embodiments. It should be understood that the embodiments are only exemplary, and should not be understood as limitation to a scope of protection of the present invention.

### Embodiments

In this embodiment, the following encoding rule 1 in Table 1 and encoding rule 2 in Table 2 are selected.

TABLE 1
Encoding rule 1

| | Output candidate | Output candidate |
|---|---|---|
| Support | base while | base while |
| bit base | input bit is 0 | input bit is 1 |
| A | T/A | G/C |
| T | G/C | T/A |
| C | T/A | G/C |
| G | G/C | T/A |

TABLE 2
Encoding rule 2

| Support | Support bit | Base | Base |
|---|---|---|---|
| bit base of | base of | combination | combination |
| encoding | encoding | while input | while input |
| rule 1 | rule 2 | bit is 0 | bit is 1 |
| A | A | T/G | A/C |
| | T | T/G | A/C |
| | C | T/G | A/C |
| | G | T/G | A/C |
| T | A | T/G | A/C |
| | T | T/G | A/C |
| | C | T/G | A/C |
| | G | T/G | A/C |
| C | A | A/C | T/G |
| | T | A/C | T/G |
| | C | A/C | T/G |
| | G | A/C | T/G |
| G | A | A/C | T/G |
| | T | A/C | T/G |
| | C | A/C | T/G |
| | G | A/C | T/G |

In this embodiment, a support bit selection mode is that the 6-th bit before a current output bit is used as the support bit of the encoding rule 1, and the first bit before the current output bit is used as the support bit of the encoding rule 2.

In this embodiment, the data selection is to convert Li Bai's “Watching the Lu Mountain Falls” into a DNA code.

Viewing the Waterfall at Mount Lu-Li Bai (Tang dynasty)

Sunlight streaming on Incense Stone kindles a violet smoke, far off I watch the waterfall plunge to the long river.

Flying waters descending straight three thousand feet, till I think the Milky Way has tumbled from the ninth height of Heaven.

The specific process is as follows:

1. Encoding and Storage

(1) A binary code corresponding to “Viewing the Waterfall at Mount Lu” is extracted, as shown in the following binary code:

11100110100110100110111110010110111010100100001110010110110001101100011110 01111000000010010001111001011011100010000011000010100000100100101101001011011110 01011001010010010000110000101111100100000111001101001110110001110111001111001 10011011110100001010111001101001011110100101111001111000010110100111111010011010 01101001100111100111100000101000100111100111100101001001111111100111101101001010 101111100111100000111001111111101111101111001000110011101001100000011010010111100 11110011100100010111110011110000000100100011110010110111000100000111110011010001 10010000010111001011000100110001101111001011011011110011101111000111000000010000 01000001010111010011010001110011110111001101011010110000001111001111001101110110 10011100100101110001000101111100100101110001000100111100101100011011000001111100 10110110000101110101110111110111100100011001110011110010110100100011110011010011 00010101111111010011001001110110110111001101011001010110011111010001001000010111 10111100100101110011001110111100101101001001010100111100011100000001000001000001 010

(2) The above binary code is split into the following two binary codes of equal length, namely a binary code 1 and a binary code 2:

Binary Code 1:

1110011010011100100110111110010110111010100100001110010110110001101100011110 01111000000010010001111001011011100010000011000010100000100100101101001011011110 01011001010010010000110000101011011100100000111001101001110110001110111001111001 10011011110100001010111001101001011110100101111001111000010110100111111010011010 01101001100111100111100000101000100111100111100101001001111111100111101101001010 101111100111100000111001111111101111101111001000110011101001100000011010010111100 1111001110010001011111001111000000010010001

Binary Code 2:

1110010110111000100000111110011010001100100000101110010110001001100011011110 01011011011110011101111000111000000010000010000010101110100110100011100111101110 01101011010110000001111001111001101110110100111001001011100010001011111001001011 10001000100111100101100011011000001111100101101100001011101011101111101111001000 11001110011110010110100100011110011010011000101011111110100110010011101101101110 01101011001010110011111010001001000010111101111001001011100110011101111001011010 01001010100111100011100000001000001000001010

(3) An initiation base sequence is selected as: “ATCAGTGCTA” (SEQ ID NO: 1), the initiation base sequence is used to provide initiation support bit information while a base is not output yet, and the initiation base sequence is an agreed virtual sequence that is only reflected in the conversion, does not appear in the final DNA sequence, and is also used in decoding.

(4) The binary code 1 and the binary code 2 are converted into a DNA encoding sequence according to the encoding rule 1 and the encoding rule 2, as shown below:

SEQ ID NO: 2:
GTACAAGGAGCATATGCACGCTTACCCTTGACTTACTGAAAGGCCGTTAC
CTCGAATTCTGCATGTTTGAGAATATGATAAATTGGACTTTCAACAAGCT
GAAACGAGTTGATCTACGCGACTGAACAATAAGACCCACACGATGAGAAC
TCCCTTAAAAGGGGAAGGTCAAAGCCGAAACTTTAACCCCTAAGCGCATG
TTTGGGATATCACTGGCTATGCCCTAAGCATGTCAGGTATTTGTCGTTCC
GAATATCGGGCTTGCAGACTATATACGCAGAGCATGATTGCACGCTTCGC
ACAGCATCGCGAGGTTTTCAGCGGGGAGCTATACTAGGGTTTCAGGGACC
CTTATCATACTTCCTGAGACCCCCACGGGCTTACCTCGCATCTATACTCT
AACCAGACAGACTCAGGAAATGAAGGCAGTCATTAGACTGCGCCCAACAG
TCCCAAAGGAAAATGCCACTATATCTCGCAACTAACATCTGCAGAGGCGC
TAACCTC

(5) The DNA encoding sequence is synthesized into DNA by a chemical synthesis method.

(6) The synthesized DNA is lyophilized into powder, and saved.

2. Read of Stored DNA Sequence

(1) A library with the stored DNA dry powder is constructed, and then a sequence thereof is obtained through a high-throughput sequencing technology, as shown below:

SEQ ID NO: 2:
GTACAAGGAGCATATGCACGCTTACCCTTGACTTACTGAAAGGCCGTTAC
CTCGAATTCTGCATGTTTGAGAATATGATAAATTGGACTTTCAACAAGCT
GAAACGAGTTGATCTACGCGACTGAACAATAAGACCCACACGATGAGAAC
TCCCTTAAAAGGGGAAGGTCAAAGCCGAAACTTTAACCCCTAAGCGCATG
TTTGGGATATCACTGGCTATGCCCTAAGCATGTCAGGTATTTGTCGTTCC
GAATATCGGGCTTGCAGACTATATACGCAGAGCATGATTGCACGCTTCGC
ACAGCATCGCGAGGTTTTCAGCGGGGAGCTATACTAGGGTTTCAGGGACC
CTTATCATACTTCCTGAGACCCCCACGGGCTTACCTCGCATCTATACTCT
AACCAGACAGACTCAGGAAATGAAGGCAGTCATTAGACTGCGCCCAACAG
TCCCAAAGGAAAATGCCACTATATCTCGCAACTAACATCTGCAGAGGCGC
TAACCTC

(2) Through the encoding rule 1 and the encoding rule 2, the above DNA sequence is decoded according to the reverse process of the encoding process, as to obtain two binary codes, namely the binary code 1 and the binary code 2:

Binary Code 1:

1110011010011100100110111110010110111010100100001110010110110001101100011110 01111000000010010001111001011011100010000011000010100000100100101101001011011110 01011001010010010000110000101011011100100000111001101001110110001110111001111001 10011011110100001010111001101001011110100101111001111000010110100111111010011010 01101001100111100111100000101000100111100111100101001001111111100111101101001010 101111100111100000111001111111101111101111001000110011101001100000011010010111100 1111001110010001011111001111000000010010001

Binary Code 2:

1110010110111000100000111110011010001100100000101110010110001001100011011110 01011011011110011101111000111000000010000010000010101110100110100011100111101110 01101011010110000001111001111001101110110100111001001011100010001011111001001011 10001000100111100101100011011000001111100101101100001011101011101111101111001000 11001110011110010110100100011110011010011000101011111110100110010011101101101110 01101011001010110011111010001001000010111101111001001011100110011101111001011010 01001010100111100011100000001000001000001010

(3) The binary code 1 and the binary code 2 are converted into text information, as shown below:

Viewing the Waterfall at Mount Lu-Li Bai (Tang dynasty)

Sunlight streaming on Incense Stone kindles a violet smoke, far off I watch the waterfall plunge to the long river.

Flying waters descending straight three thousand feet, till I think the Milky Way has tumbled from the ninth height of Heaven.

The above uses specific examples to illustrate the present invention, and it is only used to help understand the present invention, and is not used to limit the present invention. For those skilled in the art to which the present invention belongs, according to the idea of the present invention, several simple deductions, modifications or replacements may also be made.

This is a continuation of International Application No. PCT/CN2019/107426 filed on Sep. 24, 2019, the disclosure of which is incorporated herein by reference in its entirety.

### TECHNICAL FIELD

The present disclosure relates to the technical field of information storage, and in particular to an information encoding method and apparatus, an information decoding method and apparatus, a storage medium, and an information storage and interpretation method.

### BACKGROUND ART

With the development of modern technology, especially the Internet, global data shows an exponential increase. The ever-increasing amount of the data puts forward higher and higher requirements on storage technologies. Traditional storage technologies, such as tape and optical disk storage, are increasingly unable to meet the current data storage requirements due to limited storage density and time. In recent years, the development of a DNA storage technology provides a new way to solve these problems. Compared with traditional storage media, DNA as a medium for information storage has the characteristics of long storage time (it can be stored for more than thousands of years, which is more than a hundred times that of the tape and optical disk medium known by the inventor) and has a high storage density (the storage density is up to about 109 Gb/mm3, which is ten million times more than that of the tape and optical disk medium) and good storage security and the like.

DNA data storage usually includes the following steps: 1) encoding: a binary 0/1 code of computer information is converted into DNA sequence information of an A/T/C/G base; 2) synthesizing: a DNA synthesis technology is used to synthesize a corresponding DNA sequence, and an obtained DNA molecule is stored in an in vitro medium or a living cell; 3) sequencing: a sequencing technology is used to read the DNA sequence of the stored DNA molecule; and 4) decoding: a method corresponding to the encoding process in Step 1) is used to convert the DNA sequence obtained by sequencing into the binary 0/1 code, and further convert into the computer information.

In order to achieve the effective DNA data storage, it is necessary to develop a technology for the above processes. Herein, the encoding and decoding technologies involved in Steps 1) and 4) are the most critical technology for the DNA data storage. The most critical parts of this technology as follows: 1) How to maximize the density of 0/1 binary information encoded by DNA. The improvement in DNA storage density is essential to save the cost of DNA synthesis for storing information in Step 2). 2) While the 0/1 binary information is converted into the A/T/C/G base sequence, the single-base repetition, high GC and high AT between the sequences are avoided to the greatest extent. Generally speaking, continuous single-base repetition appears in the DNA sequence, both the high GC and high AT can cause difficulty in reading sequence information during the sequencing process. A conversion method of the 0/1 binary information and the A/T/C/GDNA sequence directly determines the difficulty of reading the DNA sequence during the sequencing process, and thus determines the fidelity of the data in the reading process.

At present, the most classic DNA data storage methods include a data encoding method proposed by George Church and Goldman et al. George Church et al. proposed the most primitive conversion method of binary 0/1 information to A/T/C/G base information in 2012, namely, 0 stands for A/T, 1 stands for C/G, and the encoding of 1 binary data by each nucleotide is achieved. This method can avoid the continuous single-base repetition to a certain extent, but it cannot avoid high GC or high AT in the data. While a large number of 0 or 1 repetitions appear in the data, the encoded DNA sequence may produce continuous GC or AT. At the same time, in this encoding mode, each nucleotide encodes only 1 bit of information, which is very limited.

Goldman et al. proposed a new DNA encoding method in 2013, which improved the storage density of DNA data to a certain extent. It firstly converts binary data into a ternary 0/1/2 data through Huffman coding, and then converts the 0/1/2 data into a “quaternary” A/T/C/G sequence through a designed rule. This encoding method also avoids the continuous single-base repetition, but it is still limited in the storage density of the DNA data, and its maximum coding density is 1.6 bits/base (nt). At the same time, this encoding method cannot completely solve the situation of continuous GC or AT appearing in the coding sequence. Although some other encoding methods are different from the above two methods, they cannot completely avoid the continuous GC and AT situations or the storage density is still limited.

In order to use DNA more effectively for the binary data storage, improve the density of the DNA data storage, and avoid the continuous GC and AT situations, it is particularly important to develop more effective DNA data encoding methods.

### SUMMARY

The present invention provides an information encoding method and apparatus, an information decoding method and apparatus, a storage medium, and an information storage and interpretation method. Two pieces of input binary information are converted and integrated as one piece of information, the information capacity limit is reached, and the information encoding density is high.

According to a first aspect, a method for information encoding is provided in an embodiment, including the following operations:

First binary information and second binary information as well as a first encoding rule and a second encoding rule are obtained, wherein the first encoding rule is used for encoding the first binary information, and the second encoding rule is used for encoding the second binary information.

A first output candidate symbol corresponding to a current input of the first binary information is obtained according to the first encoding rule, and a second output candidate symbol corresponding to a current input of the second binary information is obtained according to the second encoding rule, and an intersection of the first output candidate symbol and the second output candidate symbol is taken as an output symbol corresponding to a current input.

An output symbol corresponding to each binary bit of the first binary information and the second binary information is sequentially determined through the first encoding rule and the second encoding rule, as to obtain an encoding sequence formed by a plurality of the output symbols.

In a preferred embodiment, the first output candidate symbols are two of four symbols, the second output candidate symbols are two of the four symbols, and the first output candidate symbols and the second output candidate symbols have one same symbol.

The above first encoding rule is that, under a support bit of the first encoding rule, two of the four symbols are selected as the first output candidate symbols while the current input of the first binary information is 0, and the other two are used as the first output candidate symbol while the current input is 1, herein the support bit of the above first encoding rule is any one of the four symbols, and the support bit of the above first encoding rule has a first corresponding relationship with a current output bit.

The above second encoding rule is that, under the support bit of the first encoding rule and a support bit of the second encoding rule, two of the four symbols are selected as the second output candidate symbols while the current input of the second binary information is 0, the other two are used as the second output candidate symbols while the current input is 1, herein the support bit of the above second encoding rule is any one of the four symbols, and the support bit of the above second encoding rule has a second corresponding relationship with the current output bit, each support bit of the first encoding rule corresponds to four support bits of the second encoding rule.

In a preferred embodiment, a length of the above first binary information and a length of the above second binary information are equal.

In a preferred embodiment, the above first binary information and second binary information are split from a same piece of binary information.

In a preferred embodiment, the above first corresponding relationship is a first predetermined number of bits before the above current output bit; and the second corresponding relationship is a second predetermined number of bits before the current output bit.

In a preferred embodiment, the above method further includes: an initiation base sequence is obtained, and it provides the support bit for the first encoding rule and the second encoding rule before an above output base is generated.

In a preferred embodiment, the above first output candidate symbol are two base symbols of four base symbols composed of A, T, C, and G, and the above second output candidate symbol are two base symbols of the four base symbols composed of A, T, C, and G, the above support bit is one base symbol of the four base symbols composed of A, T, C, and G.

In addition, the above encoding information is a nucleic acid sequence containing the above four bases composed of A, T, C, and G.

In a preferred embodiment, the above method further includes: an initiation sequence is obtained, and it provides an initiation support bit for the first encoding rule and the second encoding rule before a first bit of the above encoding sequence is generated.

In a preferred embodiment, the above method further includes: before the first binary information and the second binary information are obtained, the above first binary information and second binary information are extracted from a computer storage device.

According to a second aspect, an apparatus for information encoding is provided in an embodiment, including an information acquiring unit, an information encoding unit, and a result generating unit.

The information acquiring unit is configured to obtain first binary information and second binary information as well as a first encoding rule and a second encoding rule, herein the first encoding rule is used for encoding the first binary information, and the second encoding rule is used for encoding the second binary information.

The information encoding unit is configured to obtain a first output candidate symbol corresponding to a current input of the first binary information according to the first encoding rule, and obtain a second output candidate symbol corresponding to a current input of the second binary information according to the second encoding rule, and take an intersection of the first output candidate symbol and the second output candidate symbol as an output symbol corresponding to the current input.

The result generating unit is configured to sequentially determine an output symbol corresponding to each binary bit of the first binary information and the second binary information through the first encoding rule and the second encoding rule, as to obtain an encoding sequence formed by a plurality of the above output symbols.

In a preferred embodiment, the above first output candidate symbols are two of four symbols, the above second output candidate symbols are two of the four symbols, and the above first output candidate symbols and the second output candidate symbols have one same symbol.

The above first encoding rule is that, under a support bit of the first encoding rule, two of the four symbols are selected as the first output candidate symbols while the current input of the first binary information is 0, and the other two are used as the first output candidate symbols while the current input is 1, herein the support bit of the above first encoding rule is any one of the four symbols, and the support bit of the above first encoding rule has a first corresponding relationship with a current output bit.

The above second encoding rule is that, under the support bit of the first encoding rule and a support bit of the second encoding rule, two of the four symbols are selected as the second output candidate s symbols while the current input of the second binary information is 0, the other two are used as the second output candidate symbols while the current input is 1, wherein the support bit of the above second encoding rule is any one of the four symbols, and the support bit of the above second encoding rule has a second corresponding relationship with the current output bit, each support bit of the first encoding rule corresponds to four support bits of the second encoding rule.

In a preferred embodiment, the above first corresponding relationship is a first predetermined number of bits before the above current output bit; and the second corresponding relationship is a second predetermined number of bits before the above current output bit.

In a preferred embodiment, the above first output candidate symbols are two base symbols of four bases composed of A, T, C, and G, and the above second output candidate symbol are two base symbols of the four base symbols composed of A, T, C, and G, the above support bit is one base symbol of the four base symbols composed of A, T, C, and G. In addition, the above encoding information is a nucleic acid sequence containing the above four bases composed of A, T, C, and G.

According to a third aspect, a computer-readable storage medium is provided in an embodiment, including a program, and the program may be executed by a processor to achieve the method as described in the first aspect.

According to a fourth aspect, a method for information storage by using a DNA sequence is provided in an embodiment, including the following operations:

Through the information encoding method as described in the first aspect, binary information to be stored is converted into DNA sequence information, and the above DNA sequence information includes a base sequence formed by four bases composed of A, T, C, and G.

A corresponding DNA sequence is synthesized according to the above DNA sequence information.

The above DNA sequence is saved to achieve the storage of information.

In a preferred embodiment, the above method further includes: The above DNA sequence information is split into multiple pieces of DNA short sequence information, and a DNA index sequence identifier is added to each piece of the split DNA short sequence information, and the above DNA index sequence identifier includes position order information of the above DNA short sequence information.

A corresponding DNA sequence is synthesized according to the above DNA short sequence information.

The above DNA sequence is saved to achieve the storage of information.

In a preferred embodiment, the above DNA sequence is saved in the form of dry powder, or saved by embedding in an embedding material.

In a preferred embodiment, the above DNA sequence is transferred into a living cell for saving.

In a preferred embodiment, the above living cell is a microbial cell, preferably Escherichia coli or Saccharomyces cerevisiae.

According to a fifth aspect, a method for information decoding is provided in an embodiment, including the following operations: An encoding sequence generated by the above encoding method as described in the first aspect, as well as a first encoding rule and a second encoding rule are obtained, herein the first encoding rule is used for encoding first binary information, and the second encoding rule is used for encoding second binary information.

A current symbol of the above encoding sequence is read, and according to a corresponding relationship between four different symbols and the binary information in the first encoding rule and the second encoding rule, the above current symbol is converted into a binary bit of the first binary information and the second binary information.

Through a corresponding relationship between the different symbols and the binary information in the first encoding rule and the second encoding rule, each binary bit of the first binary information and the second binary information corresponding to each symbol bit of the above encoding sequence is determined sequentially, and the first binary information and the second binary information with a determined binary bit order are obtained.

In a preferred embodiment, the above symbol is four base symbols composed of A, T, C, and G.

In a preferred embodiment, the above encoding sequence is obtained through the following steps (1) or (2):

(1) Each DNA sequence synthesized by the storage method as described in the fourth aspect is sequenced, as to obtain the above encoding sequence; or

(2) each DNA sequence synthesized by the storage method as described in the fourth aspect is sequenced, as to obtain each DNA short sequence information; according to a DNA index sequence identifier, position order information of each DNA short sequence is obtained; and according to the above position order information, the above each DNA short sequence is combined to form the above encoding sequence.

In a preferred embodiment, the above decoding method further includes the following operation: The above first binary information and second binary information are transcoded into corresponding information.

In a preferred embodiment, the above corresponding information is text information, image information, audio information and/or video information.

According to a sixth aspect, an apparatus for information decoding is provided in an embodiment, including an information acquiring unit, an information decoding unit and a result generating unit.

The information acquiring unit is configured to obtain an encoding sequence generated by the encoding apparatus as described in the second aspect, as well as a first encoding rule and a second encoding rule, herein the first encoding rule is used for encoding first binary information and the second encoding rule is used for encoding second binary information.

The information decoding unit is configured to read a current symbol of the above encoding sequence, and according to a corresponding relationship between different symbols and binary information in the first encoding rule and the second encoding rule, convert the above current symbol into a binary bit of the first binary information and the second binary information.

The result generating unit is configured to, through a corresponding relationship between four different symbols and the binary information in the first encoding rule and the second encoding rule, sequentially determine each binary bit of the first binary information and the second binary information corresponding to each symbol bit of the above encoding sequence, and obtain the first binary information and the second binary information with a determined binary bit order.

In a preferred embodiment, the above different symbols are four base symbols composed of A, T, C, and G respectively.

In a preferred embodiment, the above encoding sequence is obtained by the following units (1) or (2):

(1) A sequencing unit is configured to sequence each DNA sequence synthesized by the above storage method as described in the fourth aspect, as to obtain the above encoding sequence; or

(2) A sequencing unit is configured to sequence each DNA sequence synthesized by the above storage method as described in the fourth aspect, as to obtain each DNA short sequence information.

An index unit is configured to obtain position order information of above each DNA short sequence according to a DNA index sequence identifier.

A combination unit is configured to combine the above each DNA short sequence into the above encoding sequence according to the position order information.

In a preferred embodiment, the above apparatus further includes a transcoding unit. The transcoding unit is configured to transcode the above first binary information and second binary information into corresponding information.

In a preferred embodiment, the above corresponding information is text information, image information, audio information and/or video information.

According to a seventh aspect, a computer-readable storage medium is provided in an embodiment, including a program, and the program may be executed by a processor to achieve the decoding method as described in the fifth aspect.

The methods for information encoding and decoding provided by the present invention may convert and integrate two pieces of input binary information into one piece of information, the information capacity limit is reached, and the information encoding density is high. In a preferred embodiment, the four base symbols composed of A, T, C, and G are used as four different symbols to convert and integrate the two pieces of the input binary information into one DNA sequence skillfully, so that the limit of information capacity of a DNA single-base reaches 2 bits/base, and the information encoding density is high.

In practical applications, the method of the present invention may be well combined with long-segment gene storage to improve the DNA storage density. The method of the present invention utilizes two encoding rules to generate 5566277615616 encoding systems, provides a rich compilation method rule library for DNA storage applications, and greatly expands a choice space of DNA storage compilation methods.

At the same time, the encoding rule in the method of the present invention depends on the two support bits, so that the encoding mode has the higher tolerance for repeated input, and may effectively avoid or reduce the impact caused by continuous single-base and double-base repetitions. In addition, the encoding rule library generated by the method of the present invention utilizes different encoding rule combinations for encrypted storage, increases the difficulty of decrypting the DNA storage information, improves the security of the DNA storage information well, and provides the guarantee for future data security storage applications.

### BRIEF DESCRIPTION OF THE DRAWINGS

FIG. 1 is a general process diagram of DNA storage and interpretation information in an embodiment of the present invention.

FIG. 2 is a process diagram of an encoding method for converting binary information into a DNA sequence in an embodiment of the present invention.

FIG. 3 is a schematic diagram of a general representation method of an encoding rule in an embodiment of the present invention.

FIG. 4 is a schematic diagram of a method for representing a case of the encoding rule in an embodiment of the present invention.

FIG. 5 is a schematic diagram of a principle of converting one piece of binary information into a DNA sequence in an embodiment of the present invention.

FIG. 6 is a structure block diagram of an encoding apparatus for converting binary information into a DNA sequence in an embodiment of the present invention.

FIG. 7 is a process diagram of a decoding method for converting a DNA sequence into binary information in an embodiment of the present invention.

FIG. 8 is a structure block diagram of a decoding apparatus for converting a DNA sequence into binary information in an embodiment of the present invention.

FIG. 9 is a process diagram of a method for information storage by using a DNA sequence in an embodiment of the present invention.

FIG. 10 is a process diagram of a method for interpreting information stored in the form of a DNA sequence in an embodiment of the present invention.

### DETAILED DESCRIPTION OF THE EMBODIMENTS

The present invention is further described in detail below through specific embodiments in combination with drawings. In the following embodiments, many detailed descriptions are used to enable the present invention to be better understood. However, those skilled in the art may easily realize that some of features may be omitted under different circumstances, or may be replaced by other materials or methods.

In addition, the features, operations, or characteristics described in the description may be combined in any appropriate manners to form various embodiments. At the same time, various steps or actions in the method description may also be sequentially exchanged or adjusted in a manner apparent to those skilled in the art. Therefore, various orders in the description and the drawings are only for the purpose of clearly describing a certain embodiment, and are not meant to be a necessary order, unless it is specified otherwise that a certain order must be followed.

In this article, serial numbers assigned to the characteristics, such as “first”, and “second”, are only used to distinguish described objects, and do not have any order or technical meanings.

In this article, a first encoding rule is also referred to as “encoding rule 1”, and the two have equivalent meanings; a second encoding rule is also referred to as “encoding rule 2”, and the two have equivalent meanings.

As shown in FIG. 1, the data encoding, storage and interpretation processes of the present invention includes the following steps:

(1) Data Encoding and Storage Process:

Step 1: binary “0/1” computer information to be stored is extracted by using a program that comes with any computer operating systems or a program specially written to extract a binary 0/1 code.

Step 2: an encoding rule in a set of the methods for encoding and decoding in the present invention is used to convert two pieces of binary “0/1” information into a DNA sequence represented by A/T/C/G bases. Generally speaking, the two pieces of the binary “0/1” information have the same length, and the length is greater than or equal to 1.

Step 3: the DNA sequence obtained from the 0/1 binary computer information conversion is split into short fragments with a certain length, and it is convenient for the DNA synthesis in the next step.

Step 4: an index sequence (index 1 and index 2 in the figure) is added to each DNA short fragment, and the index sequence may encode order information of the short fragment obtained in Step 3.

Step 5: a DNA synthesis technology is used to synthesize the DNA sequence fragments obtained in Step 4, and save in a corresponding medium. Generally speaking, the DNA sequence may be saved in a sample tube in the form of dry powder, or saved by embedding in an embedding material such as amber, and a silicon ball, or the DNA sequence may be transferred into a living cell for saving, and the living cell may be a microbial cell, more preferably Escherichia coli or Saccharomyces cerevisiae.

(2) DNA Data Interpretation Process:

Step 6: a sequencing technology (for example, Sanger sequencing or high-throughput sequencing) is used to sequence the saved DNA fragments of storage data information, as to obtain DNA sequences of these fragments.

Step 7: according to index sequence information preset in the DNA sequences, an order of DNA fragment encoding information is interpreted, and the DNA fragments are sorted sequentially.

Step 8: according to a sequence of a DNA fragment encoding information area obtained by sorting, a decoding rule corresponding to the encoding rule in Step 2 is used to convert DNA information into 0/1 binary information.

Step 9: the 0/1 binary information obtained in Step 8 is converted into stored information (namely files, such as a text, an image, an audio or a video) by using a program that comes with any operating systems or a program specially written to convert 0/1 into data information.

As shown in FIG. 2, an encoding method for converting binary information into a DNA sequence in an embodiment of the present invention includes the following steps:

S201: first binary information and second binary information as well as a first encoding rule and a second encoding rule are obtained, herein the first encoding rule is used for encoding the first binary information, and the second encoding rule is used for encoding the second binary information.

In the embodiment of the present invention, the first binary information and the second binary information are 0/1 binary information to be encoded. Both the first binary information and the second binary information may have the same or different sources, namely, the two may be related 0/1 binary information or unrelated 0/1 binary information. In an example of the related 0/1 binary information, two pieces of the 0/1 binary information are binary information which is split from the same piece of the binary information. It is generally required that the two pieces of the 0/1 binary information have the same length, because in the method of the present invention, one binary bit (0/1) of each piece of the 0/1 binary information read every time is from a pair of binary bits (0/1) of the two pieces of the 0/1 binary information, and converted into a symbol by the method of the present invention, for example, base symbol (A, T, G, or C) information. In an example of the unrelated 0/1 binary information, for example, it is two pieces of the 0/1 binary information from text information and pattern information respectively.

In the embodiment of the present invention, the first encoding rule is an encoding rule of a corresponding relationship between a binary symbol 0/1 and an output symbol (for example, base symbols A, T, G, or C). As a typical but non-deterministic example, a case of the first encoding rule is that, under a support bit of the first encoding rule, two of the four bases composed of A, T, C, and G are selected as a first output candidate base while a current input of the first binary information is 0, and the other two are used as a first output candidate base while the current input is 1.

As shown in FIG. 3, vertical rows A, T, G, and C represent the support bits of the four different bases, and N1, N2, N3, and N4 represent one of the four bases composed of A, T, C, and G respectively; a1, a2, a3, and a4 are 0 or 1, and a1+a2+a3+a4-2; b1, b2, b3, and b4 are 0 or 1, and b1+b2+b3+b4=2; c1, c2, c3 and c4 are 0 or 1, and c1+c2+c3+c4=2; and d1, d2, d3, and d4 are 0 or 1, and d1+d2+d3+d4=2. That is to say, in a series a, there are two 1 and two 0. Similarly, there are two 1 and two 0 in series b, c, and d respectively. Therefore, the encoding rule 1 (first encoding rule) has a total of [2 of 4 combinations are chosen, namely “2 of 4 bases are optionally chosen, and each support bit corresponds to 6 possible combinations” ] to the 4-th power types, namely, 6\hat{ }4 or 1296 types, because the base selection between the 4 different support bits is independent of each other. This encoding rule is interpreted as: while the support bit is Nx (x=1, 2, 3, 4) and an input bit is 0/1, N of the two output bits corresponding to 0/1 is the possibility of output.

As shown in FIG. 4, a case of the encoding rule 1 is shown. In this case, in the case of the different support bits of the encoding rule 1, the input bit of the first binary information corresponds to a corresponding output bit in a manner shown in the figure.

In the embodiment of the present invention, the input bit refers to a binary bit of the current input of the first binary information (or the second binary information), and it is 0/1; and the output bit refers to a base bit to be output after being converted according to the first encoding rule (or the second encoding rule) corresponding to the input bit.

In the embodiment of the present invention, the support bit of the first encoding rule refers to support information needed for selecting a correct output symbol by the first encoding rule according to different inputs (the so-called “input” refers to the binary bit of the current input of the first binary information) of the first binary information. For example, while the output symbol is the base symbol A, T, C or C, the support bit of the first encoding rule is also the base symbol. Specifically, the support bit of the first encoding rule is a known base that has a first corresponding relationship with the current output bit. Generally speaking, the support bit is base information that has been converted before, for example, the number of bits before a data bit (current output bit) is being converted, such as the previous base information of the 3-th or 6-th bit. Therefore, in an embodiment of the present invention, the so-called “first corresponding relationship” refers to a first predetermined number of bits before the current output bit (for example, the number of bits set arbitrarily such as the 3-th or 6-th bit before the current input bit). Certainly, the support bit may also be virtual information generated randomly, and it has the artificially set first corresponding relationship with the current output bit. In the embodiment of the present invention, the so-called “random generation” means that a random number is generated through various random methods, and corresponds to ATCG according to a certain rule. Examples of the random methods include but are not limited to: Monte Carlo random number or U(0,1) random number and the like. In addition, the support bit may also come from a specific selection mode of a reference sequence, and it has a specific first corresponding relationship with the current output bit. For example, each output bit corresponds to a known base on the reference sequence in a specific mapping manner, such as a base order of the reference sequence corresponds to each output bit sequentially. In the embodiment of the present invention, while the support bits are different, the same input may have different base outputs.

In the embodiment of the present invention, the second encoding rule is an encoding rule of a corresponding relationship between a binary symbol 0/1 and an output symbol (for example, a base symbol A, T, G, or C). As a typical but non-deterministic example, a case of the second encoding rule is that, under the support bit of the first encoding rule and the support bit of the second encoding rule, two of the four bases are selected as second output candidate bases while an input of the second binary information is 0, and the other two are used as second output candidate bases while the input is 1; and wherein the support bit of the second encoding rule is any one of the four bases, and the support bit of the second encoding rule has a second corresponding relationship with the current output bit, and each support bit of the first encoding rule corresponds to four support bits of the second encoding rule.

As shown in FIG. 3, N01 and N02 are two possible bases while the input in the encoding rule 1 is 0, and N11 and N12 are two possible bases while the input in the encoding rule 1 is 1. Because in the encoding rule 1, the different support bits thereof may have different output possibilities, while the support bits in the encoding rule 1 are different, the corresponding encoding rule 2 (second encoding rule) may be different. Wherein xn+yn=1, xn*yn=0, n=1, 2, 3, 4, 5, 6, 7, 8. In other words, there is one 0 and one 1 in each x and y group. Therefore, corresponding to the possibility given in each encoding rule 1, there are 28 types of the encoding rule 2. This is because the base selection between the different support bits of the encoding rule 2 is independent, and the encoding rule 2 corresponding to each support bit of each encoding rule 1 has (2\hat{ }2)\hat{ }4 possibilities, namely 256 possibilities. This encoding rule is interpreted as that while the support bit is Nx (x=01, 02, 11, 12) and the input bit is 0/1, N of the two output bits corresponding to 0/1 is the possibility of output.

As shown in FIG. 4, a case of the encoding rule 2 is shown. In this case, in the case of the different support bits of the encoding rule 1, it is respectively shown that under the different support bits of the encoding rule 2, the input bit of the second binary information corresponds to a corresponding output bit in the manner shown in the figure. Specifically, it shows the corresponding encoding rule 2 while the support bit of the encoding rule 1 is A, the corresponding encoding rule 2 while the support bit of the encoding rule 1 is C, the corresponding encoding rule 2 while the support bit of the encoding rule 1 is C, and the corresponding encoding rule 2 while the support bit of the encoding rule 1 is T.

In the embodiment of the present invention, similar to the support bit of the first encoding rule, the support bit of the second encoding rule refers to support information needed for selecting a correct output symbol by the second encoding rule according to different inputs (the so-called “input” refers to the binary bit of the current input of the second binary information) of the second binary information. For example, while the output symbol is the base symbol A, T, G, or C, the support bit of the second encoding rule is also the base symbol. Specifically, the support bit of the second encoding rule is a known base that has a second corresponding relationship with the current output bit. Generally speaking, the support bit is a base that has been converted before, for example, the number of bits before a data bit (current output bit) is being converted, such as the previous base information of the 4-th or 8-th bit. Therefore, in an embodiment of the present invention, the so-called “second corresponding relationship” refers to a second predetermined number of bits before the current output bit (for example, the previous number of bits set arbitrarily such as the 4-th or 8-th bit). Certainly, the support bit may also be virtual information generated randomly, and it has the artificially set second corresponding relationship with the current output bit. In the embodiment of the present invention, the so-called “random generation” means that a random number is generated through various random methods, and corresponds to ATCG according to a certain rule. Examples of the random methods include but are not limited to: Monte Carlo random number or U(0,1) random number and the like. In addition, the support bit may also come from a specific selection mode of a reference sequence, and it has a specific second corresponding relationship with the current output bit. For example, each output bit corresponds to a known base on the reference sequence in a specific mapping manner, such as a base order of the reference sequence corresponds to each output bit sequentially. In a preferred embodiment of the present invention, the first corresponding relationship is different from the second corresponding relationship, namely the support bit of the first encoding rule and the support bit of the second encoding rule take different base bits. For example, in an embodiment of the present invention, the 6-th known base before the current output bit is selected as the support bit of the first encoding rule, and the first known base before the current output bit is selected as the support bit of the second encoding rule.

Since the base selection of the different support bits in the encoding rule 1 is independent, and the base selection of the different support bits in the encoding rule 2 is also independent, the number of the types of the encoding rule 2 corresponding to each encoding rule 1 is 256\hat{ }4, namely, 4294967296 types, so the total number of the types of this dual-encoding rule system is 1296*4294967296, namely 5566277615616, and it is about 5.6×10\hat{ }12 types.

In this way, one binary input bit (0/1) from the first binary information obtains two possible lists of output through the encoding rule 1, and one binary input bit (0/1) from the second binary information obtains two possible lists of output through the encoding rule 2, an intersection of the two output lists is taken, thereby an output base corresponding to the two input binary bits is determined.

It should be noted that in the case of using the base information that has been converted before as the support bit, while the conversion is initially started, since the base information that has been converted before as the support bit does not exist, an initial support bit problem needs to be solved by an appropriate method. In an embodiment of the present invention, the encoding method of the present invention further includes: a initiation base sequence is obtained, and it provides the support bit for the first encoding rule and the second encoding rule before the output base is generated. In other cases, for example, in the case of using the virtual information generated randomly, or the specific selection mode from the reference sequence as the support bit, such a problem does not exist.

S202: a first output candidate symbol corresponding to a current input of the first binary information is obtained according to the first encoding rule, and a second output candidate symbol corresponding to a current input of the second binary information is obtained according to the second encoding rule, and an intersection of the first output candidate symbol and the second output candidate symbol is taken as an output symbol corresponding to a current input.

In the embodiment of the present invention, as a preferred example, the first output candidate symbols and the second output candidate symbols are two base symbols in four bases composed of A, T, C, and G respectively. Therefore, the first output candidate symbol and the second output candidate symbol refer to a first output candidate base and a second output candidate base, respectively.

In the embodiment of the present invention, the so-called “current input” refers to a 0/1 binary bit currently being read from the first binary information or the second binary information, and it represents one binary data respectively. The present invention reads the 0/1 binary bits in the first binary information and the second binary information at the same time. While one 0/1 binary bit of the first binary information is read, one 0/1 binary bit of the second binary information is read at the same time. One 0/1 binary bit read of the first binary information is converted into the first output candidate base (including two bases) through the first encoding rule, and one 0/1 binary bit read of the second binary information is converted into the second output candidate base (including two bases) through the second encoding rule, and there is a common base between the first output candidate base and the second output candidate base, namely an intersection of the first output candidate base and the second output candidate base, and the intersection is the output base corresponding to the two current inputs (the current input of the first binary information and the current input of the second binary information).

S203: an output symbol corresponding to each binary bit of the first binary information and the second binary information is sequentially determined through the first encoding rule and the second encoding rule, as to obtain an encoding sequence formed by a plurality of the above output symbols.

Since the first binary information and the second binary information each have a certain length, for example, tens, hundreds, thousands, or tens of thousands of bits (0/1 binary bit), each time of the conversion operation in the above step S202 may only convert information of one bit (0/1 binary bit) in the first binary information and the second binary information into the output base respectively, so the conversion operation needs to be continuously performed, until the information of all bits (0/1 binary bit) in the first binary information and the second binary information are converted into the corresponding output bases, and a plurality of such output bases forms the corresponding DNA sequence. So far, the conversion of two pieces of the 0/1 binary information (the first binary information and the second binary information) into a piece of the DNA sequence information is completed.

In order to enable the present invention to be understood more easily, as a typical but non-deterministic example, as shown in FIG. 5: there are two pieces of binary information a and b, and a 0/1 binary information encoding sequence thereof is shown in FIG. 5. A virtual sequence shown in the figure is used as a initiation base support bit, the ninth binary bit 1 of the binary information a is converted into a G/C base while the support bit is A according to the first encoding rule (Table 1); and while the support bit of the ninth binary bit 1 of the binary information a is A, and the support bit of the ninth binary bit 1 of the binary information b is A, the ninth binary bit 1 of the binary information b is converted into a A/C base while the support bit is A according to the second encoding rule (Table 2), and an intersection of the G/C base and the A/C base is taken, namely the base C is used as the output base.

Those skilled in the art may understand that all or part of the functions of the various methods in the above embodiments may be achieved in the form of hardware, or achieved in the form of a computer program. While all or part of the functions in the above embodiments is achieved in the form of the computer program, the program may be stored in a computer-readable storage medium. The storage medium may include: a read-only memory, a random access memory, a magnetic disk, an optical disk, a hard disk and the like. The program is executed by a computer to achieve the above functions. For example, the program is stored in a memory of a device, and while the program in the memory is executed by a processor, all or part of the above functions may be achieved. In addition, when all or part of the functions in the above embodiments is achieved in the form of the computer program, the program may also be stored in the storage medium such as a server, another computer, a magnetic disk, an optical disk, a flash disk, or a mobile hard disk, and saved in a memory of a local device by downloading or copying, or the version of a system of the local device is updated, while the program in the memory is executed by the processor, all or part of the functions in the above embodiments may be achieved.

Therefore, corresponding to the information encoding method of the present invention, an embodiment of the present invention further provides an apparatus for information encoding. As shown in FIG. 6, the apparatus includes: an information acquiring unit 601, configured to obtain first binary information and second binary information as well as a first encoding rule and a second encoding rule, herein the first encoding rule is used for encoding the first binary information, and the second encoding rule is used for encoding the second binary information; an information encoding unit 602, configured to obtain a first output candidate symbol corresponding to a current input of the first binary information according to the first encoding rule, and obtain a second output candidate symbol corresponding to a current input of the second binary information according to the second encoding rule, and take an intersection of the first output candidate symbol and the second output candidate symbol as an output symbol corresponding to the current input; and a result generating unit 603, configured to sequentially determine an output symbol corresponding to each binary bit of the first binary information and the second binary information through the first encoding rule and the second encoding rule, as to obtain an encoding sequence formed by a plurality of the above output symbols.

In a preferred embodiment of the present invention, the above first output candidate symbols and second output candidate symbols are two base symbols of four bases composed of A, T, C, and G respectively; the above first encoding rule is that, under a support bit of the first encoding rule, two of the four bases composed of A, T, C, and G are selected as the first output candidate base while the current input of the first binary information is 0, and the other two are used as the first output candidate base while the current input is 1; the second encoding rule is that, under the support bit of the first encoding rule and a support bit of the second encoding rule, two of the four bases are selected as the second output candidate base while the current input of the second binary information is 0, the other two are used as the second output candidate base while the current input is 1; and wherein the support bit of the first encoding rule is a known base that has a first corresponding relationship with the current output bit, the support bit of the second encoding rule is a known base that has a second corresponding relationship with the current output bit, and each support bit of the first encoding rule corresponds to four support bits of the second encoding rule.

Corresponding to the information encoding method of the present invention, an embodiment of the present invention further provides a computer-readable storage medium, including a program, and the program may be executed by a processor to achieve the information encoding method as described in the present invention.

As an inverse process of the encoding method of the present invention, an embodiment of the present invention further provides an information decoding method. As shown in FIG. 7, the method includes:

S701: an encoding sequence generated by the above encoding method, as well as a first encoding rule and a second encoding rule are obtained, herein the first encoding rule is used for encoding first binary information, and the second encoding rule is used for encoding second binary information.

In the embodiment of the present invention, the encoding sequence generated by the encoding method, for example, may be a piece of DNA sequence information. Correspondingly, as a typical but non-deterministic example, the first encoding rule is that: under the support bit of the first encoding rule, two of the four bases composed of A, T, C, and G are selected as the first output candidate bases while the input of the first binary information is 0, and the other two are used as the first output candidate bases while the input is 1.

As a typical but non-deterministic example, in the embodiment of the present invention, the second encoding rule is that: under the support bit of the first encoding rule and the support bit of the second encoding rule, two of the four bases are selected as the second output candidate bases while the input of the second binary information is 0, and the other two are used as the second output candidate bases while the input is 1.

In the embodiment of the present invention, the support bit of the first encoding rule is a known base that has a first corresponding relationship with the current output bit, and the support bit of the second encoding rule is a known base that has a second corresponding relationship with the current output bit, and each support bit of the first encoding rule corresponds to four support bits of the second encoding rule.

S702: a current symbol of the above information encoded by four different symbols is read, and according to a corresponding relationship between the four different symbols and the binary information in the first encoding rule and the second encoding rule, the above current symbol is converted into a binary bit of the first binary information and the second binary information.

As a typical but non-deterministic example, in the embodiment of the present invention, the four bases composed of A, T, C, and G are used as four different encoding symbols. Correspondingly, the current symbol is also called “current base”. The so-called “current base” refers to a base on a DNA sequence that is currently read and converted into the binary bit. Since there are tens, hundreds, thousands, or even tens of thousands of the bases in the DNA sequence, each base being currently read and converted is the so-called “current base”.

S703: through a corresponding relationship between the different symbols and the binary information in the first encoding rule and the second encoding rule, each binary bit of the first binary information and the second binary information corresponding to each symbol bit of the above encoding sequence is determined sequentially, and the first binary information and the second binary information with a determined binary bit order are obtained.

In an embodiment of the present invention, the above encoding sequence is obtained through the following steps (1) or (2).

(1) Each DNA sequence synthesized by the storage method of the present invention is sequenced, as to obtain the above encoding sequence; or

(2) each DNA sequence synthesized by the storage method of the present invention is sequenced, as to obtain each DNA short sequence information; according to a DNA index sequence identifier, position order information of each DNA short sequence is obtained; and according to the above position order information, the above each DNA short sequence is combined to form the above encoding sequence.

In an embodiment of the present invention, the above decoding method further includes: the above first binary information and second binary information are transcoded into corresponding information, for example, text information, image information, audio information and/or video information.

It should be noted that many details in the above decoding method, especially the details related to the technical features such as the first binary information, the second binary information, the first encoding rule, the second encoding rule, the input bit, the output bit, and the support bit, are the same as the details of such technical features in the above encoding method, so they are not repeatedly described.

Corresponding to the information decoding method of the present invention, an embodiment of the present invention further provides an apparatus for information decoding. As shown in FIG. 8, the apparatus includes: an information acquiring unit 801, configured to obtain an encoding sequence generated by the above encoding apparatus, as well as a first encoding rule and a second encoding rule, herein the first encoding rule is used for encoding first binary information, and the second encoding rule is used for encoding second binary information; an information decoding unit 802, configured to read a current symbol of the above encoding sequence, and according to a corresponding relationship between different symbols and binary information in the first encoding rule and the second encoding rule, convert the above current symbol into a binary bit of the first binary information and the second binary information; and a result generating unit 803, configured to, through a corresponding relationship between four different symbols and the binary information in the first encoding rule and the second encoding rule, sequentially determine each binary bit of the first binary information and the second binary information corresponding to each symbol bit of the above encoding sequence, and obtain the first binary information and the second binary information with a determined binary bit order.

In an embodiment of the present invention, the above encoding sequence is obtained by the following units (1) or (2).

(1) A sequencing unit is configured to sequence each DNA sequence synthesized by the storage method of the present invention, as to obtain the above encoding sequence; or

(2) a sequencing unit is configured to sequence each DNA sequence synthesized by the storage method of the present invention, as to obtain each DNA short sequence information; an index unit is configured to obtain position order information of above each DNA short sequence according to a DNA index sequence identifier; and a combination unit is configured to combine the above each DNA short sequence into the above encoding sequence according to the position order information.

In an embodiment of the present invention, the above decoding apparatus further includes a transcoding unit. The transcoding unit is configured to transcode the above first binary information and second binary information into corresponding information, for example, text, image, audio or video information.

In an embodiment of the present invention, the above different symbols are the four base symbols composed of A, T, C, and G; and similarly, many details in the above decoding device, especially the details related to the technical features such as the first binary information, the second binary information, the first encoding rule, the second encoding rule, the input bit, the output bit and the support bit, are the same as the details of such technical features in the above encoding method, so they are not repeatedly described.

Corresponding to the information decoding method of the present invention, an embodiment of the present invention further provides a computer-readable storage medium, including a program, and the program may be executed by a processor to achieve the information decoding method as described in the present invention.

An embodiment of the present invention further provides a method for information storage by using a DNA sequence. As shown in FIG. 9, the method includes the following operations.

S901: through the information encoding method of the present invention, binary information to be stored is converted into DNA sequence information, and the DNA sequence information includes a base sequence formed by four bases composed of A, T, C, and G.

S902: a corresponding DNA sequence is synthesized according to the above DNA sequence information.

S903: the above DNA sequence is saved to achieve the storage of information.

An embodiment of the present invention further provides a method for interpreting information stored in the form of a DNA sequence. As shown in FIG. 10, the method includes the following operations.

S1001: a DNA fragment for storing data information is obtained.

S1002: a DNA sequence of the above DNA fragment is obtained by sequencing.

S1003: through the decoding method for converting the DNA sequence into the binary information, the above DNA sequence is converted into the binary information.

In some embodiments, the DNA sequence information converted from the binary information is longer, and it is not beneficial to direct synthesis. Therefore, as a preferred method, the DNA sequence information is split into a plurality of pieces of DNA short sequence information, and a DNA index sequence identifier is added to each piece of the split DNA short sequence information, and the above DNA index sequence identifier contains position order information of the above DNA short sequence information; then, the corresponding DNA sequence is synthesized according to the above DNA short sequence information; and finally, the above DNA sequence is saved to achieve the storage of information.

Generally speaking, the DNA sequence may be saved in a sample tube in the form of dry powder, or saved by embedding in an embedding material such as amber, and a silicon ball, or the DNA sequence may be transferred into a living cell for saving, and the living cell may be a microbial cell, more preferably Escherichia coli or Saccharomyces cerevisiae.

In some embodiments, there are a plurality of pieces of the DNA fragments for storing the data information, and each piece of the DNA fragment has a corresponding index sequence as the encoding order information of the DNA fragment. In this case, in order to obtain the complete binary information, the DNA sequence obtained by sequencing needs to be sorted firstly according to the index sequence to obtain a complete DNA sequence after sorting, and then the complete DNA sequence is converted into the binary information through the decoding method of the present invention for converting the DNA sequence into the binary information.

The technical schemes of the present invention are described in detail below through the embodiments. It should be understood that the embodiments are only exemplary, and should not be understood as limitation to a scope of protection of the present invention.

### Embodiments

In this embodiment, the following encoding rule 1 in Table 1 and encoding rule 2 in Table 2 are selected.

TABLE 1
Encoding rule 1

| | Output candidate | Output candidate |
|---|---|---|
| Support | base while | base while |
| bit base | input bit is 0 | input bit is 1 |
| A | T/A | G/C |
| T | G/C | T/A |
| C | T/A | G/C |
| G | G/C | T/A |

TABLE 2
Encoding rule 2

| Support bit | Support bit | Base | Base |
|---|---|---|---|
| base of | base of | combination | combination |
| encoding | encoding | while input | while input |
| rule 1 | rule 2 | bit is 0 | bit is 1 |
| A | A | T/G | A/C |
| | T | T/G | A/C |
| | C | T/G | A/C |
| | G | T/G | A/C |
| T | A | T/G | A/C |
| | T | T/G | A/C |
| | C | T/G | A/C |
| | G | T/G | A/C |
| C | A | A/C | T/G |
| | T | A/C | T/G |
| | C | A/C | T/G |
| | G | A/C | T/G |
| G | A | A/C | T/G |
| | T | A/C | T/G |
| | C | A/C | T/G |
| | G | A/C | T/G |

In this embodiment, a support bit selection mode is that the 6-th bit before a current output bit is used as the support bit of the encoding rule 1, and the first bit before the current output bit is used as the support bit of the encoding rule 2.

In this embodiment, the data selection is to convert Li Bai's “Watching the Lu Mountain Falls” into a DNA code.

Viewing the Waterfall at Mount Lu-Li Bai (Tang dynasty)

Sunlight streaming on Incense Stone kindles a violet smoke, far off I watch the waterfall plunge to the long river.

Flying waters descending straight three thousand feet, till I think the Milky Way has tumbled from the ninth height of Heaven.

The specific process is as follows:

1. Encoding and Storage

(1) A binary code corresponding to “Viewing the Waterfall at Mount Lu” is extracted, as shown in the following binary code:

11100110100110100110111110010110111010100100001110010110110001101100011110 01111000000010010001111001011011100010000011000010100000100100101101001011011110 01011001010010010000110000101111100100000111001101001110110001110111001111001 10011011110100001010111001101001011110100101111001111000010110100111111010011010 01101001100111100111100000101000100111100111100101001001111111100111101101001010 101111100111100000111001111111101111101111001000110011101001100000011010010111100 11110011100100010111110011110000000100100011110010110111000100000111110011010001 10010000010111001011000100110001101111001011011011110011101111000111000000010000 01000001010111010011010001110011110111001101011010110000001111001111001101110110 10011100100101110001000101111100100101110001000100111100101100011011000001111100 10110110000101110101110111110111100100011001110011110010110100100011110011010011 00010101111111010011001001110110110111001101011001010110011111010001001000010111 10111100100101110011001110111100101101001001010100111100011100000001000001000001 010

(2) The above binary code is split into the following two binary codes of equal length, namely a binary code 1 and a binary code 2:

Binary Code 1:

1110011010011100100110111110010110111010100100001110010110110001101100011110 01111000000010010001111001011011100010000011000010100000100100101101001011011110 01011001010010010000110000101011011100100000111001101001110110001110111001111001 10011011110100001010111001101001011110100101111001111000010110100111111010011010 01101001100111100111100000101000100111100111100101001001111111100111101101001010 101111100111100000111001111111101111101111001000110011101001100000011010010111100 1111001110010001011111001111000000010010001

Binary Code 2:

1110010110111000100000111110011010001100100000101110010110001001100011011110 01011011011110011101111000111000000010000010000010101110100110100011100111101110 01101011010110000001111001111001101110110100111001001011100010001011111001001011 10001000100111100101100011011000001111100101101100001011101011101111101111001000 11001110011110010110100100011110011010011000101011111110100110010011101101101110 01101011001010110011111010001001000010111101111001001011100110011101111001011010 01001010100111100011100000001000001000001010

(3) An initiation base sequence is selected as: “ATCAGTGCTA” (SEQ ID NO: 1), the initiation base sequence is used to provide initiation support bit information while a base is not output yet, and the initiation base sequence is an agreed virtual sequence that is only reflected in the conversion, does not appear in the final DNA sequence, and is also used in decoding.

(4) The binary code 1 and the binary code 2 are converted into a DNA encoding sequence according to the encoding rule 1 and the encoding rule 2, as shown below:

SEQ ID NO: 2:
GTACAAGGAGCATATGCACGCTTACCCTTGACTTACTGAAAGGCCGTTAC
CTCGAATTCTGCATGTTTGAGAATATGATAAATTGGACTTTCAACAAGCT
GAAACGAGTTGATCTACGCGACTGAACAATAAGACCCACACGATGAGAAC
TCCCTTAAAAGGGGAAGGTCAAAGCCGAAACTTTAACCCCTAAGCGCATG
TTTGGGATATCACTGGCTATGCCCTAAGCATGTCAGGTATTTGTCGTTCC
GAATATCGGGCTTGCAGACTATATACGCAGAGCATGATTGCACGCTTCGC
ACAGCATCGCGAGGTTTTCAGCGGGGAGCTATACTAGGGTTTCAGGGACC
CTTATCATACTTCCTGAGACCCCCACGGGCTTACCTCGCATCTATACTCT
AACCAGACAGACTCAGGAAATGAAGGCAGTCATTAGACTGCGCCCAACAG
TCCCAAAGGAAAATGCCACTATATCTCGCAACTAACATCTGCAGAGGCGC
TAACCTC

(5) The DNA encoding sequence is synthesized into DNA by a chemical synthesis method.

(6) The synthesized DNA is lyophilized into powder, and saved.

2. Read of Stored DNA Sequence

(1) A library with the stored DNA dry powder is constructed, and then a sequence thereof is obtained through a high-throughput sequencing technology, as shown below:

SEQ ID NO: 2:
GTACAAGGAGCATATGCACGCTTACCCTTGACTTACTGAAAGGCCGTTAC
CTCGAATTCTGCATGTTTGAGAATATGATAAATTGGACTTTCAACAAGCT
GAAACGAGTTGATCTACGCGACTGAACAATAAGACCCACACGATGAGAAC
TCCCTTAAAAGGGGAAGGTCAAAGCCGAAACTTTAACCCCTAAGCGCATG
TTTGGGATATCACTGGCTATGCCCTAAGCATGTCAGGTATTTGTCGTTCC
GAATATCGGGCTTGCAGACTATATACGCAGAGCATGATTGCACGCTTCGC
ACAGCATCGCGAGGTTTTCAGCGGGGAGCTATACTAGGGTTTCAGGGACC
CTTATCATACTTCCTGAGACCCCCACGGGCTTACCTCGCATCTATACTCT
AACCAGACAGACTCAGGAAATGAAGGCAGTCATTAGACTGCGCCCAACAG
TCCCAAAGGAAAATGCCACTATATCTCGCAACTAACATCTGCAGAGGCGC
TAACCTC

(2) Through the encoding rule 1 and the encoding rule 2, the above DNA sequence is decoded according to the reverse process of the encoding process, as to obtain two binary codes, namely the binary code 1 and the binary code 2:

Binary Code 1:

1110011010011100100110111110010110111010100100001110010110110001101100011110 01111000000010010001111001011011100010000011000010100000100100101101001011011110 01011001010010010000110000101011011100100000111001101001110110001110111001111001 10011011110100001010111001101001011110100101111001111000010110100111111010011010 01101001100111100111100000101000100111100111100101001001111111100111101101001010 101111100111100000111001111111101111101111001000110011101001100000011010010111100 1111001110010001011111001111000000010010001

Binary Code 2:

1110010110111000100000111110011010001100100000101110010110001001100011011110 01011011011110011101111000111000000010000010000010101110100110100011100111101110 01101011010110000001111001111001101110110100111001001011100010001011111001001011 10001000100111100101100011011000001111100101101100001011101011101111101111001000 11001110011110010110100100011110011010011000101011111110100110010011101101101110 01101011001010110011111010001001000010111101111001001011100110011101111001011010 01001010100111100011100000001000001000001010

(3) The binary code 1 and the binary code 2 are converted into text information, as shown below:

Viewing the Waterfall at Mount Lu-Li Bai (Tang dynasty)

Sunlight streaming on Incense Stone kindles a violet smoke, far off I watch the waterfall plunge to the long river.

Flying waters descending straight three thousand feet, till I think the Milky Way has tumbled from the ninth height of Heaven.

The above uses specific examples to illustrate the present invention, and it is only used to help understand the present invention, and is not used to limit the present invention. For those skilled in the art to which the present invention belongs, according to the idea of the present invention, several simple deductions, modifications or replacements may also be made.

1. A method for information encoding, wherein the method comprises:
acquiring first binary information and second binary information, as well as a first encoding rule and a second encoding rule, wherein the first encoding rule is used for encoding the first binary information, and the second encoding rule is used for encoding the second binary information;
acquiring a first output candidate symbol corresponding to a current input of the first binary information according to the first encoding rule, and acquiring a second output candidate symbol corresponding to a current input of the second binary information according to the second encoding rule, and taking an intersection of the first output candidate symbol and the second output candidate symbol, as an output symbol corresponding to a current input; and
sequentially determining an output symbol corresponding to each binary bit of the first binary information and the second binary information through the first encoding rule and the second encoding rule, as to obtain an encoding sequence formed by a plurality of the output symbols.

2. The method according to claim 1, wherein the first output candidate symbols are two of four symbols, the second output candidate symbols are two of the four symbols, and the first output candidate symbols and the second output candidate symbols have one same symbol;
the first encoding rule is that, under a support bit of the first encoding rule, two of the four symbols are selected as the first output candidate symbols while the current input of the first binary information is 0, and the other two are used as the first output candidate symbols while the current input is 1, wherein the support bit of the first encoding rule is any one of the four symbols, and the support bit of the first encoding rule has a first corresponding relationship with a current output bit; and
the second encoding rule is that, under the support bit of the first encoding rule and a support bit of the second encoding rule, two of the four symbols are selected as the second output candidate symbols while the current input of the second binary information is 0, the other two are used as the second output candidate symbols while the current input is 1, wherein the support bit of the second encoding rule is any one of the four symbols, and the support bit of the second encoding rule has a second corresponding relationship with the current output bit, each support bit of the first encoding rule corresponds to four support bits of the second encoding rule.

3. The method according to claim 1, wherein a length of the first binary information and a length of the second binary information are equal.

4. The method according to claim 3, wherein the first binary information and the second binary information are split from a same piece of binary information.

5. The method according to claim 1, wherein the first corresponding relationship is a first predetermined number of bits before the current output bit; and the second corresponding relationship is a second predetermined number of bits before the current output bit.

6. The method according to claim 1, wherein the first output candidate symbols are two base symbols of four bases composed of A, T, C, and G, and the second output candidate symbols are two base symbols of the four bases composed of A, T, C, and G, the support bit is one base symbol of the four bases composed of A, T, C, and G; and
the encoding information is a nucleic acid sequence containing the four bases composed of A, T, C, and G.

7. The method according to claim 1, wherein the method further comprises:
acquiring an initiation sequence, which provides an initiation support bit for the first encoding rule and the second encoding rule before a first bit of the encoding sequence is generated.

8. The method according to claim 1, wherein the method further comprises:
before acquiring the first binary information and the second binary information, extracting the first binary information and the second binary information from a computer storage device.

9. (canceled)

10. (canceled)

11. (canceled)

12. (canceled)

13. (canceled)

14. A method for information storage by using a DNA sequence, wherein the method comprises:
through the information encoding method according to any one of claim 1, converting binary information to be stored into DNA sequence information, wherein the DNA sequence information comprises a base sequence formed by four bases composed of A, T, C, and G;
synthesizing a corresponding DNA sequence according to the DNA sequence information; and
saving the DNA sequence to achieve the storage of information.

15. The method according to claim 14, wherein the method further comprises:
splitting the DNA sequence information into multiple pieces of DNA short sequence information, and adding a DNA index sequence identifier to each piece of the split DNA short sequence information, wherein the DNA index sequence identifier comprises position order information of the DNA short sequence information;
synthesizing a corresponding DNA sequence according to the DNA short sequence information; and
saving the DNA sequence to achieve the storage of information.

16. The method according to claim 14, wherein the DNA sequence is saved in the form of dry powder, or saved by embedding in an embedding material.

17. The method according to claim 14, wherein the DNA sequence is transferred into a living cell for saving.

18. The method according to claim 17, wherein the living cell is a microbial cell, preferably Escherichia coli or Saccharomyces cerevisiae.

19. A method for information decoding, wherein the method comprises:
acquiring an encoding sequence generated by the encoding method according to any one of claim 1, as well as a first encoding rule and a second encoding rule, wherein the first encoding rule is used for encoding first binary information, and the second encoding rule is used for encoding second binary information;
reading a current symbol of the encoding sequence, and according to a corresponding relationship between different symbols and the binary information in the first encoding rule and the second encoding rule, converting the current symbol into a binary bit of the first binary information and the second binary information; and
through a corresponding relationship between the different symbols and the binary information in the first encoding rule and the second encoding rule, sequentially determining each binary bit of the first binary information and the second binary information corresponding to each symbol bit of the encoding sequence, and acquiring the first binary information and the second binary information with a determined binary bit order.

20. The method according to claim 19, wherein the symbol is four base symbols composed of A, T, C, and G.

21. The method according to claim 20, wherein the encoding sequence is obtained through the following steps (1) or (2):
(1) sequencing each DNA sequence synthesized by the storage method according to claim 14, as to obtain the encoding sequence; or
(2) sequencing each DNA sequence synthesized by the storage method according to claim 15, as to obtain each DNA short sequence information;
according to a DNA index sequence identifier, acquiring position order information of each DNA short sequence; and
according to the position order information, combining the each DNA short sequence to form the encoding sequence.

22. The method according to claim 19, wherein the method further comprises:
transcoding the first binary information and the second binary information into corresponding information.

23. The method according to claim 22, wherein the corresponding information is text information, image information, audio information and/or video information.

24. (canceled)

25. (canceled)

26. (canceled)

27. (canceled)

28. (canceled)

29. (canceled)

30. The method according to claim 2, wherein a length of the first binary information and a length of the second binary information are equal.

31. The method according to claim 2, wherein the first corresponding relationship is a first predetermined number of bits before the current output bit; and the second corresponding relationship is a second predetermined number of bits before the current output bit.


SEQUENCE LISTING (2 sequences)

SEQ ID NO: 1 - DNA, 10 bp, Artificial Sequence
  initiation base sequence
atcagtgcta

SEQ ID NO: 2 - DNA, 507 bp, Artificial Sequence
  DNA encoding sequence
gtacaaggagcatatgcacgcttacccttgacttactgaaaggccgttacctcgaattctgcatgtttgagaatatgataaattggactttcaacaagctgaaacgagttgatctacgcgactgaacaataagacccacacgatgagaactcccttaaaaggggaaggtcaaagccgaaactttaacccctaagcgcatgtttgggatatcactggctatgccctaagcatgtcaggtatttgtcgttccgaatatcgggcttgcagactatatacgcagagcatgattgcacgcttcgcacagcatcgcgaggttttcagcggggagctatactagggtttcagggacccttatcatacttcctgagacccccacgggcttacctcgcatctatactctaaccagacagactcaggaaatgaaggcagtcattagactgcgcccaacagtcccaaaggaaaatgccactatatctcgcaactaacatctgcagaggcgctaacctc